(19) United States
(12) Patent Application Publication
(10) Pub. No.: US 2020/0375507 A1
(43) Pub. Date: Dec. 3, 2020

(54) BEHAVIOR DETERMINATION DEVICE, BEHAVIOR DETERMINATION SYSTEM, BEHAVIOR DETERMINATION METHOD, AND COMPUTER-READABLE STORAGE MEDIUM

(71) Applicant: Ochanomizu University, Tokyo (JP)

(72) Inventors: Emi ANZAI, Tokyo (JP); Yuji OHTA, Tokyo (JP); Dian REN, Tokyo (JP)

(21) Appl. No.: 16/999,400

(22) Filed: Aug. 21, 2020

Related U.S. Application Data
(63) Continuation of application No. PCT/JP2019/005873, filed on Feb. 18, 2019.

(30) Foreign Application Priority Data
Feb. 26, 2018 (JP) 2018-032336

(51) Int. Cl. A61B 5/11; A61B 5/103; A61B 5/00; G16H 40/67; G16H 50/30; G16H 50/70; A43B 3/00; A43B 13/14

(52) U.S. Cl. CPC A61B 5/1123; A61B 5/1038; A61B 5/7278; A61B 5/6807; G16H 40/67; A61B 2562/0247; G16H 50/70; A43B 3/0005; A43B 13/14; A61B 2562/04; G16H 50/30

(57) ABSTRACT

A behavior determination device includes a measurement data receiving device configured to acquires measurement data indicating a pressure or a force measured by one or a plurality of sensors provided on a sole surface of a user's foot, a data analyzing device configured to analyze the measurement data, to identify one load phase in which the user makes one step, and calculate a plantar pressure parameter and a time parameter for every one load phase, and a behavior determining device configured to detect a peak point where a maximum local maximum is obtained for every predetermined time, based on the plantar pressure parameter and the time parameter, and determine a behavior of the user based on the peak point.

### CROSS-REFERENCE TO RELATED APPLICATIONS

This application is a continuation application of International Application No. PCT/JP2019/005873 filed on Feb. 18, 2019, and designated the U.S., which is based upon and claims priority to Japanese Patent Application No. 2018-032336, filed on Feb. 26, 2018, the entire contents of which are incorporated herein by reference.

### BACKGROUND OF THE INVENTION

### 1. Field of the Invention

The present invention relates to a behavior determination device, a behavior determination system, a behavior determination method, and a computer-readable storage medium.

### 2. Description of the Related Art

Techniques for monitoring biometric information of users utilizing the so-called Internet of Things (IoT) technology are known.

For example, a system first detects a pressure distribution of a sole using pressure sensors. There is a known method that then determines a state of the user from among a standing state, a sitting state, a walking state, a brisk walking state, a fast walking state, and a running state, as described in Japanese Laid-Open Patent Publication No. 2011-138530, for example.

However, the conventional method may not be able to accurately determine the behavior of the user.

### SUMMARY OF THE INVENTION

Accordingly, it is one object of the embodiments of the present invention to accurately determine the behavior of the user.

According to one aspect of the embodiments, a behavior determination device includes a measurement data receiving device configured to acquires measurement data indicating a pressure or a force measured by one or a plurality of sensors provided on a sole surface of a user's foot; a data analyzing device configured to analyze the measurement data, to identify one load phase in which the user makes one step, and calculate a plantar pressure parameter and a time parameters for every one load phase; and a behavior determining device configured to detect a peak point where a maximum local maximum is obtained for every predetermined time, based on the plantar pressure parameter and the time parameter, and determine a behavior of the user based on the peak point.

Other objects and further features of the present invention will be apparent from the following detailed description when read in conjunction with the accompanying drawings.

### BRIEF DESCRIPTION OF THE DRAWINGS

FIG. 1 is a functional block diagram illustrating an example of a system configuration.

FIG. 2 is a diagram (part 1) illustrating an example of data.

FIG. 3 is a diagram (part 2) illustrating the example of the data.

FIG. 4 is a diagram (part 3) illustrating the example of the data.

FIG. 5 is a diagram (part 4) illustrating the example of the data.

FIG. 6 is a diagram (part 4) illustrating the example of the data.

FIG. 7 is a diagram illustrating an example of a layout of sensor positions.

FIG. 8 is a block diagram illustrating an example of a hardware configuration related to information processing performed by an information processing device such as a measuring device, an information teLminal, a server device, a management terminal, or the like.

FIG. 9 is a flow chart illustrating an example of an overall process.

FIG. 10A and FIG. 10B are diagrams illustrating an example of measurement data.

FIG. 11 is a diagram illustrating an example of plantar pressure parameter acquisition during a single-leg support phase.

FIG. 12 is a diagram illustrating the example of the plantar pressure parameter acquisition during the single-leg support phase.

FIG. 13 is a diagram illustrating an example of time parameter acquisition during the single-leg support phase.

FIG. 14 is a diagram illustrating an example of measurement data of four behavior types.

FIG. 15 is a flow chart illustrating an example of a behavior deteLmination process.

FIG. 16 is a diagram illustrating an example of 1 load phase data measured from a walking behavior.

FIG. 17 is a diagram illustrating an example of 1 load phase data measured from a cycling behavior.

FIG. 18 is a diagram illustrating an example of 1 load phase data measured from a stair climbing down behavior.

FIG. 19 is a diagram illustrating an example of 1 load phase data measured from a stair climbing up behavior.

FIG. 20 is a diagram (part 1) illustrating an example of determining the running behavior.

FIG. 21 is a diagram (part 2) illustrating the example of determining the running behavior.

FIG. 22 is a diagram illustrating an example of a peak difference between first and second phases of 1 load phase.

FIG. 23 is a diagram illustrating an example of a peak value of each sensor for each behavior.

FIG. 24 is a flow chart illustrating a modification of the behavior determination process.

### DESCRIPTION OF THE PREFERRED EMBODIMENTS

Suitable embodiments of the present invention will be described in the following, with reference to the accompanying drawings.

<Example of System Configuration>

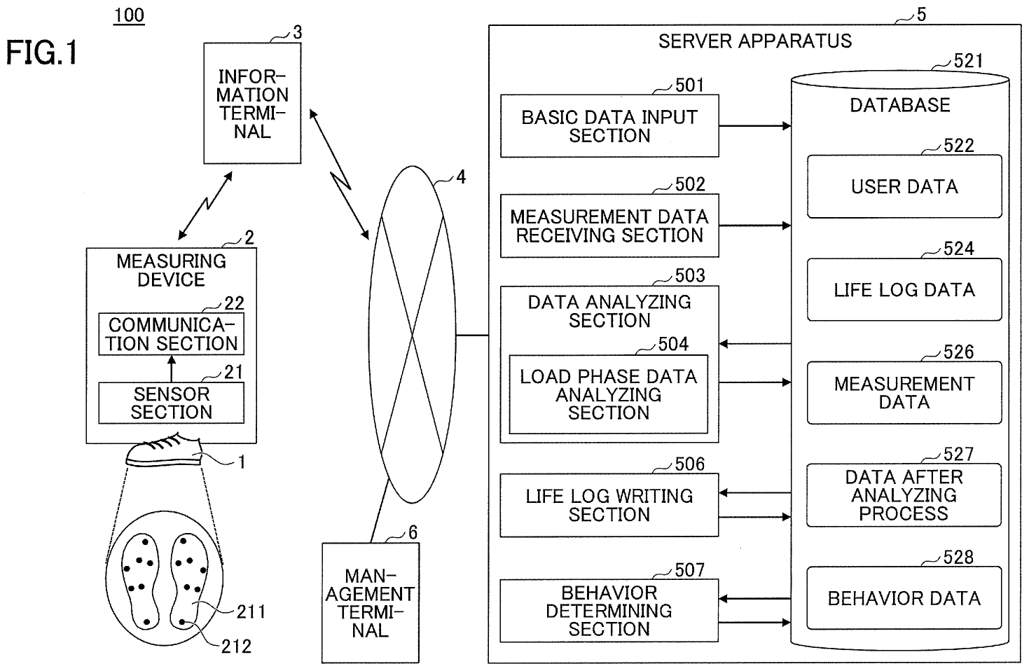

FIG. 1 is a functional block diagram illustrating an example of a system configuration. For example, a behavior determination system 100 includes a measuring device (shoe device) 2, an information terminal 3, a server device 5, or the like. The behavior determination system 100 may further include an information processing apparatus, such as a management terminal 6 or the like, as illustrated in FIG. 1. In the following, the behavior determination system 100 illustrated in FIG. 1 will be described as an example. In the example of the behavior determination system 100 illustrated in FIG. 1, the server device 5 becomes a behavior determination device. Although the server device 5 is described as an example of the behavior determination device in the following, the behavior determination device may be used in a form other than that illustrated in FIG. 1.

In the behavior determination system 100, as illustrated in FIG. 1, shoes 1 (left and right) used by the user are provided with a measuring device (shoe device) 2.

As illustrated in FIG. 1, the measuring device 2 has a functional configuration including a sensor section (or device) 21 and a communication section (or device) 22.

The measuring device 2 first measures the pressure at a sole surface of the user's feet, by the sensor section 21 thereof. Alternatively, the sensor section 21 may measure the force at the sole surface of the user's feet.

Next, the communication section 22 transmits measurement data measured by the sensor section 21 to the information terminal 3 by wireless communication, such as BLUETOOTH (registered trademark), wireless Local Area Network (LAN), or the like.

The information terminal 3 may be an information processing device, such as a smartphone, a tablet, a Personal Computer (PC), an arbitrary combination thereof, or the like, for example.

The measuring device 2 transmits the measurement data to the information terminal 3 for every 10 milliseconds (ms, or at 100 Hz), for example. In this manner, the measuring device 2 transmits the measurement data to the information terminal 3 at predetermined intervals set in advance.

The sensor section 21 may be formed by one or more pressure sensors 212 or the like, provided in a so-called insole type substrate 211 or the like, for example. The pressure sensor 212 is not limited to being provided in the insole. For example, the pressure sensor 212 may be provided in socks, shoe soles, or the like.

A sensor other than the pressure sensor 212, such as a shear force (frictional force) sensor, an acceleration sensor, a temperature sensor, a humidity sensor, an arbitrary combination thereof, or the like, may be used in place of the pressure sensor 212.

Further, the insole may be provided with a mechanism for causing a color change (mechanism for applying stimulus to sense of vision), or a mechanism for causing material deformation or change in material hardness (mechanism for applying stimulus to sense of touch), under a control from the information terminal 3.

The information terminal 3 may be provided with a feedback of the state of the walking or feet to be indicated to the user. Moreover, the communication section 22 may transmit position data or the like, using a Global Positioning System (GPS) or the like. The position data may be acquired by the information terminal 3.

The information terminal 3 transmits the measurement data received from the measuring device 2 to the server device 5 via a network 4, such as the Internet, at predetermined intervals (for example, for every 10 seconds or the like) set in advance.

In addition, the information terminal 3 may include functions such as acquiring data indicating a state of the user's walking, foot portion, or the like from the server device 5 and displaying the data on a screen, to feed back the state of the user's walking, foot portion, or the like, or to assist in the selection of shoes.

The measurement data or the like may be transmitted from the measuring device 2 directly to the server device 5. In this case, the information terminal 3 is used for performing operations with respect to the measuring device 2, making a feedback to the user, or the like, for example.

The server device 5 has a functional configuration including a basic data input section 501, a measurement data receiving section 502, a data analyzing section 503, a behavior determining section 507, and a database 521, for example. The server device 5 may have a functional configuration including a life log writing section 506 or the like, as illustrated in FIG. 1. As an example, the server device 5 described in the following is assumed to have the functional configuration illustrated in FIG. 1, however, the server device 5 is not limited to the functional configuration illustrated in FIG. 1.

The basic data input section 501 performs a basic data input procedure for receiving (or accepting) basic data settings such as the user, the shoes, or the like. For example, the setting received by the basic data input section 501 is registered in user data 522 or the like of a database 521.

The measurement data receiving section 502 performs a measurement data receiving procedure for receiving the data or the like transmitted from the measuring device 2 via the infoLmation terminal 3. The measurement data receiving section 502 registers the received data in measurement data 526 or the like of the database 521.

The data analyzing section 503 includes a load phase data analyzing section 504 or the like. For example, the load phase data analyzing section 504 performs a data analyzing procedure for analyzing the measurement data 526 and generating data after analyzing process, 527 (hereinafter also referred to as “post-analysis data 527”) or the like.

The life log writing section 506 registers life log data 524 in the database 521.

The behavior determining section 507 performs a behavior determining procedure for determining the user's behavior (including movement, action, or the like) by a behavior determining process or the like.

An administrator may access the server device 5 through the network 4 by the management terminal 6 or the like. The administrator may check the data managed by the server device 5, perform maintenance, or the like.

As illustrated in FIG. 1, the database 521 stores data including the user data 522, the life log data 524, the measurement data 526, the post-analysis data 527, behavior data 528, or the like, for example. Each of these data may be configured as follows, for example.

<Example of Data>

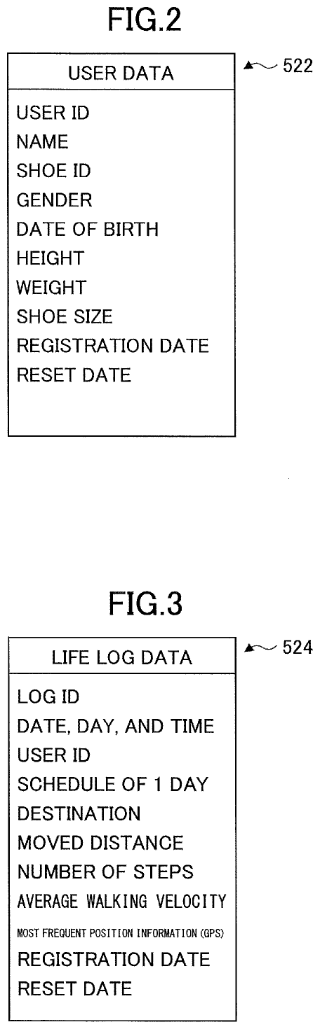

FIG. 2 is a diagram (part 1) illustrating an example of the data.

User data 522 includes items such as “user identification (ID)”, “name”, “shoe ID”, “gender”, “date of birth”, “height”, “weight”, “shoe size”, “registration date”, “reset date”, or the like, as illustrated in the FIG. 2. In other words, the user data 522 is the data for inputting features or the like of the user.

FIG. 3 is a diagram (Part 2) illustrating the example of the data.

The life log data 524 includes items such as “log ID”, “date, day, and time”, “user ID”, “schedule of 1 day”, “destination”, “moved distance”, “number of steps”, “average walking velocity”, “most frequent position information (GPS)”, “registration date”, “reset date”, or the like, as illustrated in FIG. 3. In other words, the life log data 524 is the data indicating the user's behavior (which may include the schedule).

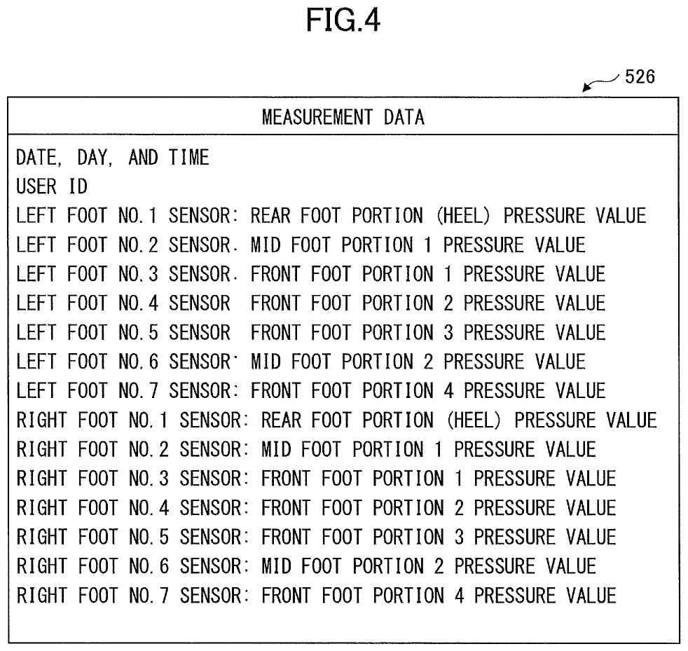

FIG. 4 is a diagram (Part 3) illustrating the example of the data.

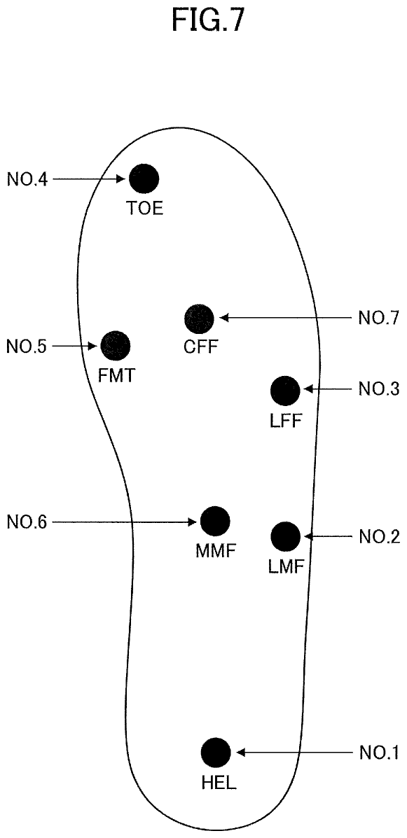

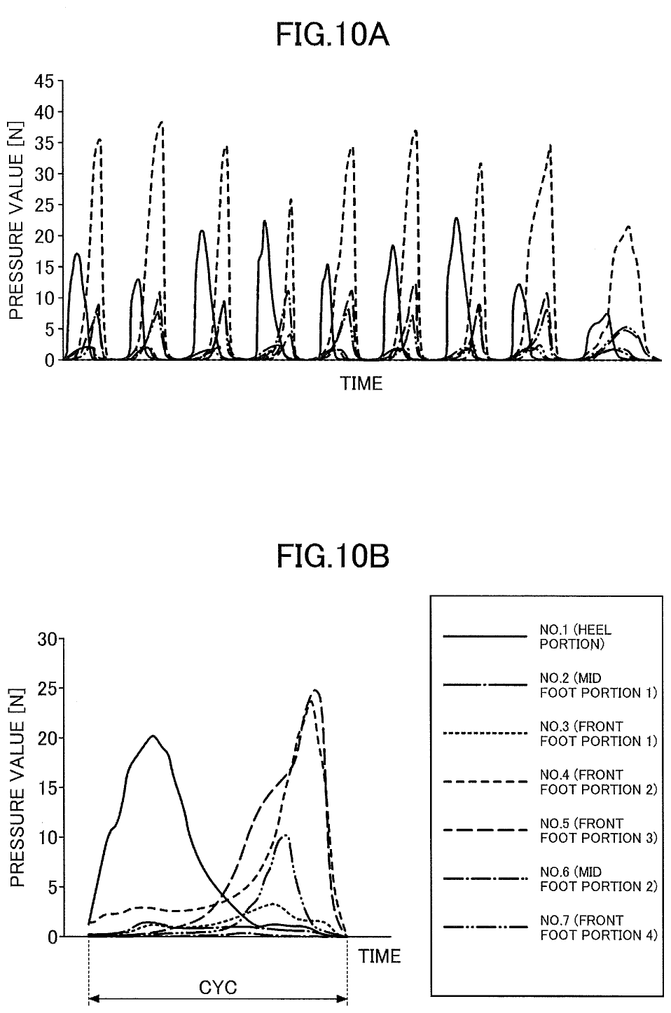

The measurement data 526 includes items such as “date, day, and time”, “user ID”, “left foot No. 1 sensor: rear foot portion (heel) pressure value”, “left foot No. 2 sensor: mid foot portion 1 pressure value”, “left foot No. 3 sensor: front foot portion 1 pressure value”, “left foot No. 4 sensor: front foot portion 2 pressure value”, “left foot No. 5 sensor: front foot portion 3 pressure value”, “left foot No. 6 sensor: mid foot portion 2 pressure value”, “left foot No. 7 sensor: front foot portion 4 pressure value”, “right foot No. 1 sensor: rear foot portion (heel) pressure value”, “right foot No. 2 sensor: mid foot portion 1 pressure value”, “right foot No. 3 sensor: front foot portion 1 pressure value”, “right foot No. 4 sensor: front foot portion 2 pressure value”, “right foot No. 5 sensor: front foot portion 3 pressure value”, “right foot No. 6 sensor: mid foot portion 2 pressure value”, “right foot No. 7 sensor: front foot portion 4 pressure value”, or the like, as illustrated in FIG. 4. An example of a specific layout of each sensor will be described later in conjunction with FIG. 7 or the like. In addition, each pressure value of the measurement data 526 may have a format of waveform data plotted during measured time. An example of the waveform data will be described later in conjunction with FIG. 10A and FIG. 10B, or the like.

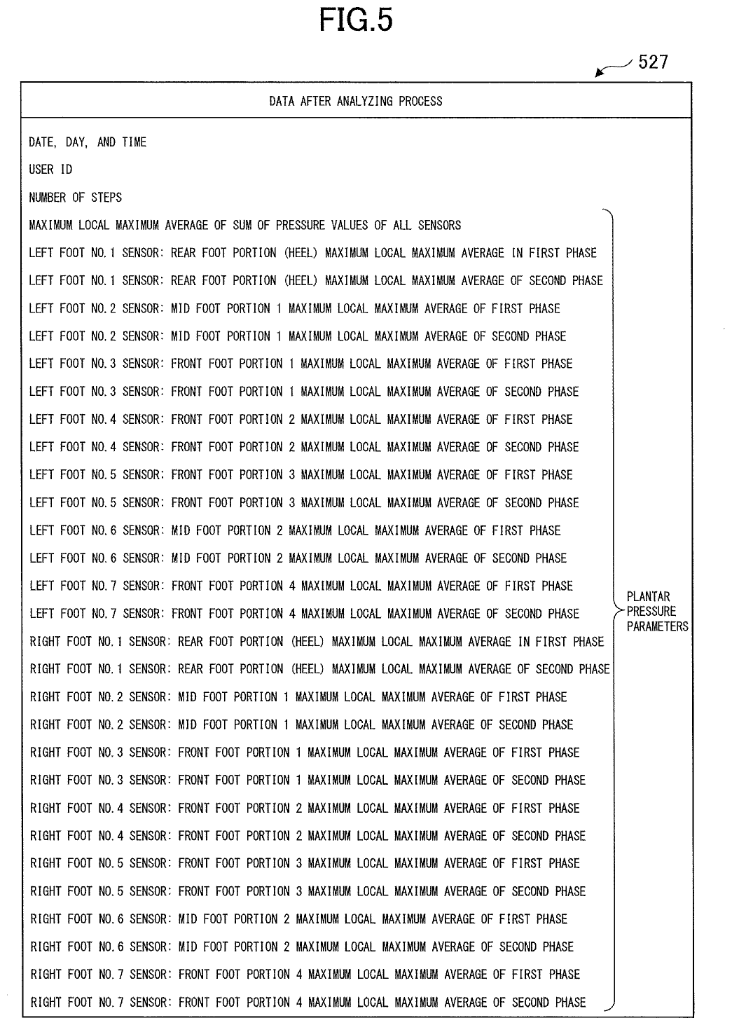

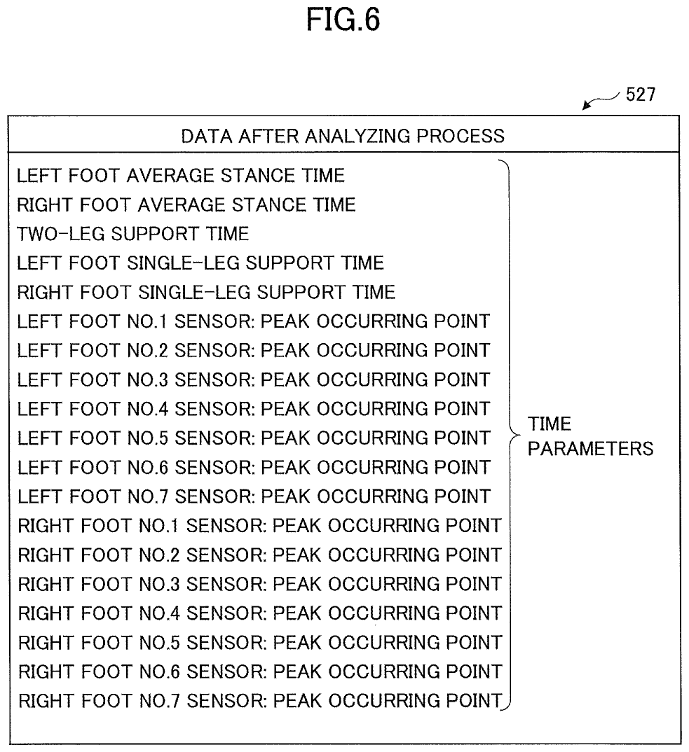

FIG. 5 and FIG. 6 are diagrams (Part 4) illustrating the example of the data.

The post-analysis data 527 includes items, such as “date, day, and time”, “user ID”, “number of steps”, “maximum local maximum average of sum of pressure values of all sensors (including a maximum local maximum average of sum of pressure values of all sensors provided with respect to the left foot, and a maximum local maximum average of sum of pressure values of all sensors provided with respect to the right foot)”, “left foot No. 1 sensor: rear foot portion (heel) maximum local maximum average in first phase”, “left foot No. 1 sensor: rear foot portion (heel) maximum local maximum average of second phase”, “left foot No. 2 sensor: mid foot portion maximum local maximum average of first phase”, “left foot No. 2 sensor: mid foot portion maximum local maximum average of second phase”, “left foot No. 3 sensor: front foot portion 1 maximum local maximum average of first phase”, “left foot No. 3 sensor: front foot portion 1 maximum local maximum average of second phase”, “left foot No. 4 sensor: front foot portion 2 maximum local maximum average of first phase”, “left foot No. 4 sensor: front foot portion 2 maximum local maximum average of second phase”, “left foot No. 5 sensor: front foot portion 3 maximum local maximum average of first phase”, “left foot No. 5 sensor: front foot portion 3 maximum local maximum average of second phase”, “left foot No. 6 sensor: mid foot portion 2 maximum local maximum average of first phase”, “left foot No. 6 sensor: mid foot portion 2 maximum local maximum average of second phase”, “left foot No. 7 sensor: front foot portion 4 maximum local maximum average of first phase”, “left foot No. 7 sensor: front foot portion 4 maximum local maximum average of second phase”, “right foot No. 1 sensor: rear foot portion (heel) maximum local maximum average in first phase”, “right foot No. 1 sensor: rear foot portion (heel) maximum local maximum average of second phase”, “right foot No. 2 sensor: mid foot portion maximum local maximum average of first phase”, “right foot No. 2 sensor: mid foot portion maximum local maximum average of second phase”, “right foot No. 3 sensor: front foot portion 1 maximum local maximum average of first phase”, “right foot No. 3 sensor: front foot portion 1 maximum local maximum average of second phase”, “right foot No. 4 sensor: front foot portion 2 maximum local maximum average of first phase”, “right foot No. 4 sensor: front foot portion 2 maximum local maximum average of second phase”, “right foot No. 5 sensor: front foot portion 3 maximum local maximum average of first phase”, “right foot No. 5 sensor: front foot portion 3 maximum local maximum average of second phase”, “right foot No. 6 sensor: mid foot portion 2 maximum local maximum average of first phase”, “right foot No. 6 sensor: mid foot portion 2 maximum local maximum average of second phase”, “right foot No. 7 sensor: front foot portion 4 maximum local maximum average of first phase”, “right foot No. 7 sensor: front foot portion 4 maximum local maximum average of second phase”, or the like, as illustrated in FIG. 5. The post-analysis data 527 further includes “left foot average stance time”, “right foot average stance time”, “two-leg support time”, “left foot single-leg support time”, “right foot single-leg support time”, “left foot No. 1 sensor: peak occurring point”, “left foot No. 2 sensor: peak occurring point”, “left foot No. 3 sensor: peak occurring point”, “left foot No. 4 sensor: peak occurring point”, “left foot No. 5 sensor: peak occurring point”, “left foot No. 6 sensor: peak occurring point”, “left foot No. 7 sensor: peak occurring point”, “right foot No. 1 sensor: peak occurring point”, “right foot No. 2 sensor: peak occurring point”, “right foot No. 3 sensor: peak occurring point”, “right foot No. 4 sensor: peak occurring point”, “right foot No. 5 sensor: peak occurring point”, “right foot No. 6 sensor: peak occurring point”, “right foot No. 7 sensor: peak occurring point”, or the like, as illustrated in FIG. 6.

Examples of time parameters of the post-analysis data 527 include “left foot average stance time”, “right foot average stance time”, “two-leg support time”, “left foot single-leg support time”, “right foot single-leg support time”, “left foot No. 1 sensor: peak occurring point”, “left foot No. 2 sensor: peak occurring point”, “left foot No. 3 sensor: peak occurring point”, “left foot No. 4 sensor: peak occurring point”, “left foot No. 5 sensor: peak occurring point”, “left foot No. 6 sensor: peak occurring point”, “left foot No. 7 sensor: peak occurring point”, “right foot No. 1 sensor: peak occurring point”, “right foot No. 2 sensor: peak occurring point”, “right foot No. 3 sensor: peak occurring point”, “right foot No. 4 sensor: peak occurring point”, “right foot No. 5 sensor: peak occurring point”, “right foot No. 6 sensor: peak occurring point”, “right foot No. 7 sensor: peak occurring point”, or the like, as illustrated in FIG. 6.

Examples of the plantar pressure parameters of the post-analysis data 527 include “maximum local maximum average of sum of pressure values of all sensors”, “left foot No. 1 sensor: rear foot portion (heel) maximum local maximum average in first phase”, “left foot No. 1 sensor: rear foot portion (heel) maximum local maximum average of second phase”, “left foot No. 2 sensor: mid foot portion maximum local maximum average of first phase”, “left foot No. 2 sensor: mid foot portion maximum local maximum average of second phase”, “left foot No. 3 sensor: front foot portion 1 maximum local maximum average of first phase”, “left foot No. 3 sensor: front foot portion 1 maximum local maximum average of second phase”, “left foot No. 4 sensor: front foot portion 2 maximum local maximum average of first phase”, “left foot No. 4 sensor: front foot portion 2 maximum local maximum average of second phase”, “left foot No. 5 sensor: front foot portion 3 maximum local maximum average of first phase”, “left foot No. 5 sensor: front foot portion 3 maximum local maximum average of second phase”, “left foot No. 6 sensor: mid foot portion 2 maximum local maximum average of first phase”, “left foot No. 6 sensor: mid foot portion 2 maximum local maximum average of second phase”, “left foot No. 7 sensor: front foot portion 4 maximum local maximum average of first phase”, “left foot No. 7 sensor: front foot portion 4 maximum local maximum average of second phase”, “right foot No. 1 sensor: rear foot portion (heel) maximum local maximum average in first phase”, “right foot No. 1 sensor: rear foot portion (heel) maximum local maximum average of second phase”, “right foot No. 2 sensor: mid foot portion maximum local maximum average of first phase”, “right foot No. 2 sensor: mid foot portion maximum local maximum average of second phase”, “right foot No. 3 sensor: front foot portion 1 maximum local maximum average of first phase”, “right foot No. 3 sensor: front foot portion 1 maximum local maximum average of second phase”, “right foot No. 4 sensor: front foot portion 2 maximum local maximum average of first phase”, “right foot No. 4 sensor: front foot portion 2 maximum local maximum average of second phase”, “right foot No. 5 sensor: front foot portion 3 maximum local maximum average of first phase”, “right foot No. 5 sensor: front foot portion 3 maximum local maximum average of second phase”, “right foot No. 6 sensor: mid foot portion 2 maximum local maximum average of first phase”, “right foot No. 6 sensor: mid foot portion 2 maximum local maximum average of second phase”, “right foot No. 7 sensor: front foot portion 4 maximum local maximum average of first phase”, “right foot No. 7 sensor: front foot portion 4 maximum local maximum average of second phase”, or the like, as illustrated in FIG. 5.

The behavior data 528 is the data indicating the determination result of the user' behavior determined by the behavior determining section 507. In other words, the behavior data 528 holds information indicating the types of user's behavior.

The user data 522 and the life log data 524 are not essential data. In addition, each data described above does not necessarily have to include all of the items illustrated in FIG. 2 through FIG. 6.

<Example of Sensor Layout>

FIG. 7 is a diagram illustrating an example of a layout of sensor positions. For example, the sensors may be provided at the positions illustrated in FIG. 7. As illustrated in FIG. 7, the sensors are desirably provided at a plurality so that the front, the mid, and the rear portions of the user's foot can be measured, respectively.

In the layout example illustrated in FIG. 7, “No. 1 sensor” or the like measures the rear portion and generates the measurement data. In other words, the sensor provided at a rear portion HEL is an example of a sensor for measuring the rear portion at the sole surface. In addition, the sensor provided at the rear portion HEL is mainly targeted to measure a range called the “rear foot portion” which includes the heel or the like.

Further, in the layout example illustrated in FIG. 7, “No. 2 sensor”, “No. 6 sensor”, or the like measure the mid portion and generate the measurement data. In other words, the sensors provided at a mid portion LMF and a mid portion MMF are examples of the sensors for measuring the mid portion at the sole surface. In addition, the sensors provided at the mid portion LMF and the mid portion MMF are mainly targeted to measure a range called the “mid foot portion”.

Further, in the layout example illustrated in FIG. 7, the “No. 3 sensor”, “No. 4 sensor”, “No. 5 sensor”, “No. 7 sensor”, or the like measure the front portion and generate measurement data. In other words, sensors provided at a front portion LFF, a front portion TOE, a front portion FMT, a front portion CFF, or the like are examples of the sensors for measuring the front portion at the sole surface. In addition, the sensors provided at the front portion LFF, the front portion TOE, the front portion FMT, and the front portion CFF are mainly targeted to measure a ranged called the “front foot portion”.

The sensor may be located at positions other than the positions illustrated in FIG. 7.

<Example Hardware Configuration>

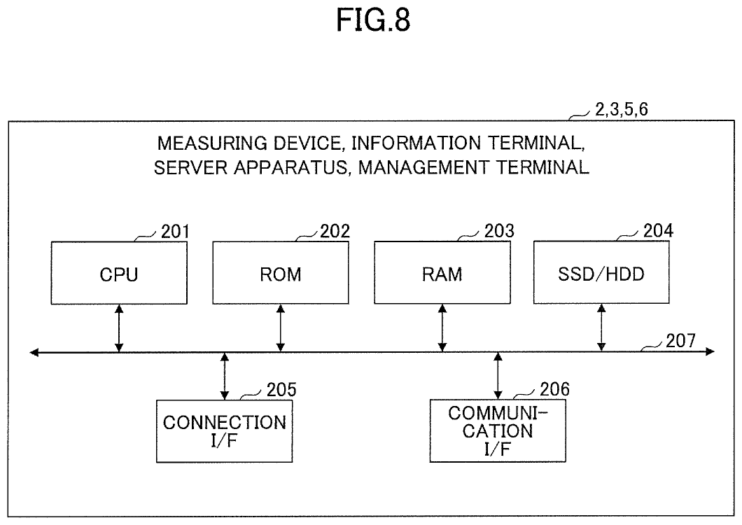

FIG. 8 is a block diagram illustrating an example of a hardware configuration related to information processing performed by an information processing device, such as a measuring device, an information terminal, a server device, a management terminal, or the like. As illustrated in FIG. 8, the information processing device, such as the measuring device, the information terminal, the server device, the management terminal, or the like is a general-purpose computer, for example. Hereinafter, an example will be described for a case where each information processing device has the same hardware configuration, however, each information processing device may have a different hardware configuration.

The measuring device 2 or the like includes a Central Processing Unit (CPU) 201, a Read Only Memory (ROM) 202, a Random Access Memory (RAM) 203, and a Solid State Drive (SSD)/Hard Disk Drive (HDD) 204 that are connected to each other via a bus 207. The ROM 202, the RAM 203, and the SSD/HDD 204 may form a computer-readable storage medium. In addition, the measuring device 2 or the like include an input device and an output device, such as a connection interface (I/F) 205, a communication I/F 206, or the like.

The CPU 201 is an example of an arithmetic unit and a control unit. It is possible to perform each process and each control by executing a program stored in an auxiliary storage device, such as the ROM 202, the SSD/HDD 204, or the like, using a main storage device, such as the RAM 203 or the as a work area. Each function of the measuring device 2 or the like is realized by executing a predetermined program in the CPU 201, for example. The program may be acquired through a computer-readable storage medium, acquired through a network or the like, or may be input in advance to the ROM 202, or the like.

According to the hardware configuration illustrated in FIG. 8, the measurement data receiving section 502, for example, may be formed by the connection I/F 205, the communication I/F 206, or the like. The data analyzing section 503 and the behavior determining section 507, for example, may be formed by the CPU 201, or the like.

<Example of Overall Process>

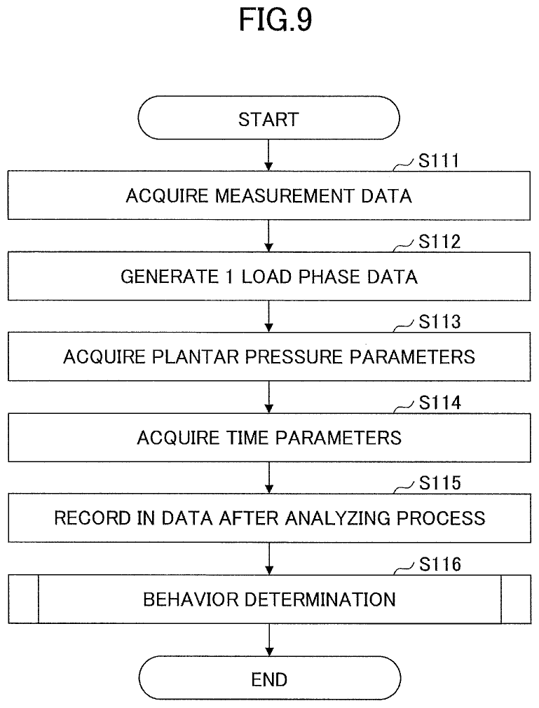

FIG. 9 is a flow chart illustrating an example of an overall process.

<Example of Acquiring Measurement Data> (Step S111)

In step S111, the behavior determination device acquires the measurement data. More particularly, in the system configuration illustrated in FIG. 1, the server device 5 acquires, via the information terminal 3 or the like, the measurement data that is generated by the measurement performed by the measuring device 2. Details of the measurement data will be described later in conjunction with FIG. 10A and FIG. 10B.

<Example of Generating of 1 Load Phase Data> (Step S112)

In step S112, the behavior determination device generates 1 load phase data. In other words, the behavior determination device analyzes and identifies the range of the measurement data, which corresponds to 1 load phase of each behavior, and generates the “1 load phase data”.

The 1 load phase corresponds to a time for making 1 step in a behavior such as walking, running, or the like, making 1 step in a behavior such as climbing up stairs, climbing down stairs, or the like, or making 1 step on a pedal (that is, 1 pedal-pressing step) in a behavior such as cycling (that is, riding a bicycle). Accordingly, when the user is walking, 1 load phase is 1 stance time (or phase).

For example, the behavior determination device first extracts a time from a rise of the waveform to a point in time when contact is made with the ground, based on the waveform data time-sequentially including the pressure value of each sensor indicated by the measurement data. The behavior determination device identifies the 1 load phase in this manner. Next, the behavior determination device extracts the waveform data for every 1 load phase.

More particularly, the behavior determination device identifies the 1 load phase, from a point where the pressure values of all of the sensors become a minimum to a point where the pressure values of all of the sensors next become a minimum. In addition, 1 load phase data is generated so as to satisfy the following conditions (a) and (b), for example.

(a) When the sensor that indicates a highest pressure value (top maximum value) in the entire waveform data is identified, a maximum local maximum of each of a plurality of load phases in the waveform data from the identified sensor indicates a value of 80% or greater with reference to the top maximum value.

(b) A length of 1 load phase is less than 1200 ms.

Further, details of the 1 load phase data will be described later in conjunction with FIG. 10A and FIG. 10B.

<Example of Acquiring of Plantar Pressure Parameter> (Step S113)

In step S113, the behavior determination device acquires plantar pressure parameters. More particularly, the behavior determination device first detects the maximum local maximum of the pressure value measured by each sensor, or the maximum local maximum of a sum of pressure values of all of the sensors of one foot, for each of a first phase and a second phase of 1 load phase. Next, the behavior determination device adds the detected maximum local maximums. By dividing a total value, obtained by the adding of the detected maximum local maximums, by the number of load phases, the behavior determination device can calculate an average value. The average value calculated in this manner is a value such as “Left foot No. 1 sensor: rear foot portion (heel) maximum local maximum average in first phase”, “maximum local maximum average of sum of pressure values of all sensors provided with respect to the left foot”, or the like.

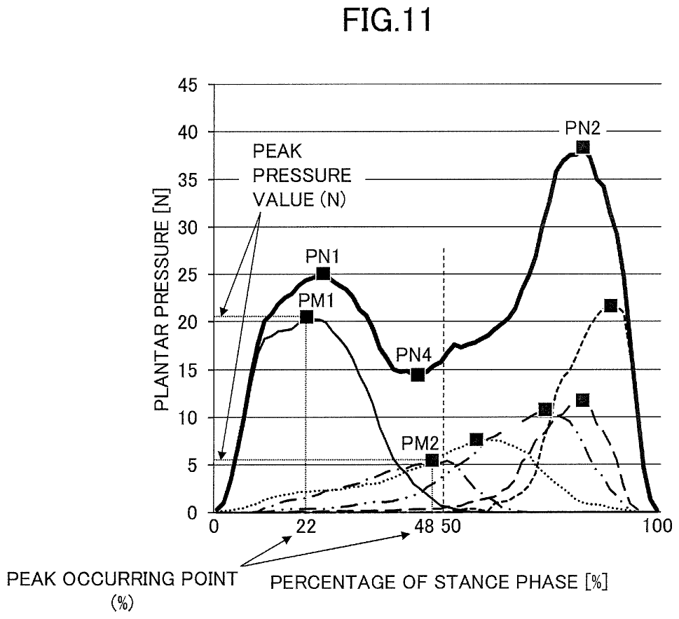

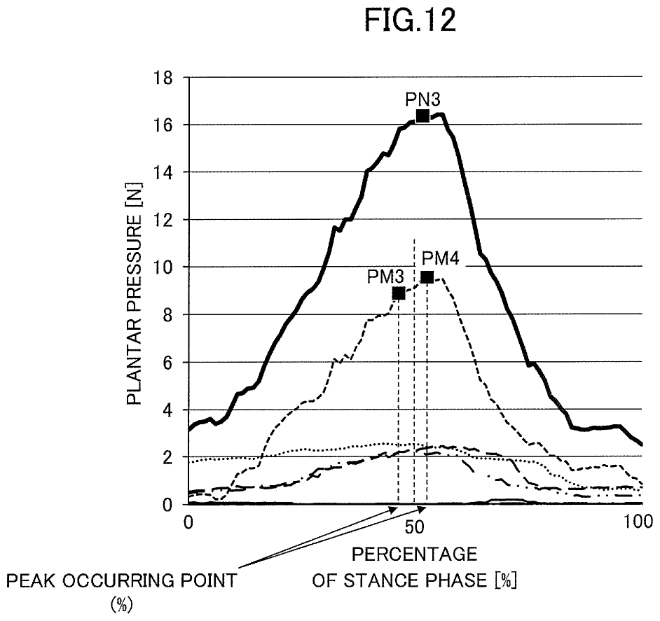

Details of the plantar pressure parameters will be described later in conjunction with FIG. 11 and FIG. 12.

<Example of Acquiring Time Parameters> (Step S114)

In step S114, the behavior determination device acquires time parameters. More particularly, the behavior determination device identifies a single-leg support phase or the like of each foot, based on the time during which each foot makes contact with the ground or the like. In addition, the behavior determination device identifies a two-leg support phase or the like of each foot, based on the time during which both feet make contact with the ground or the like. Further, the behavior determination device may calculate an average value or the like, by averaging the times for each of these loading phases, to obtain each of the time parameters. Moreover, the behavior determination device acquires the point in time when the maximum local maximum of each sensor is calculated for every first phase and second phase of 1 load phase, in a time ratio by regarding the length of 1 load phase as “100”, and regards the acquired point in time as a peak occurring point or the like, for example.

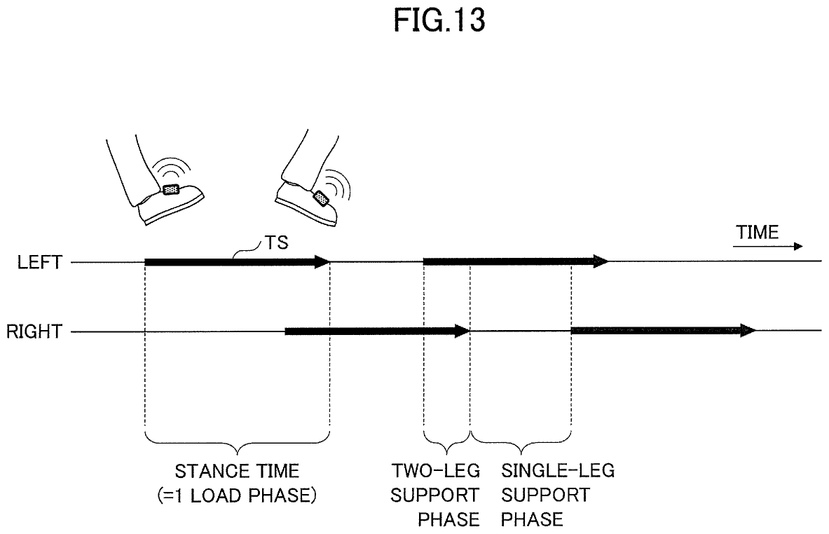

Details of the time parameters will be described later in conjunction with FIG. 11, FIG. 12, and FIG. 13.

<Example of Recording Data after Analyzing Process> (Step S115)

In step S115, the behavior determination device records the results of the analyzing performed in step S113, step S114, or the like in the post-analysis data. For example, the post-analysis data are recorded using the items or the like illustrated in FIG. 5 and FIG. 6.

<Example of Behavior Determination> (Step S116)

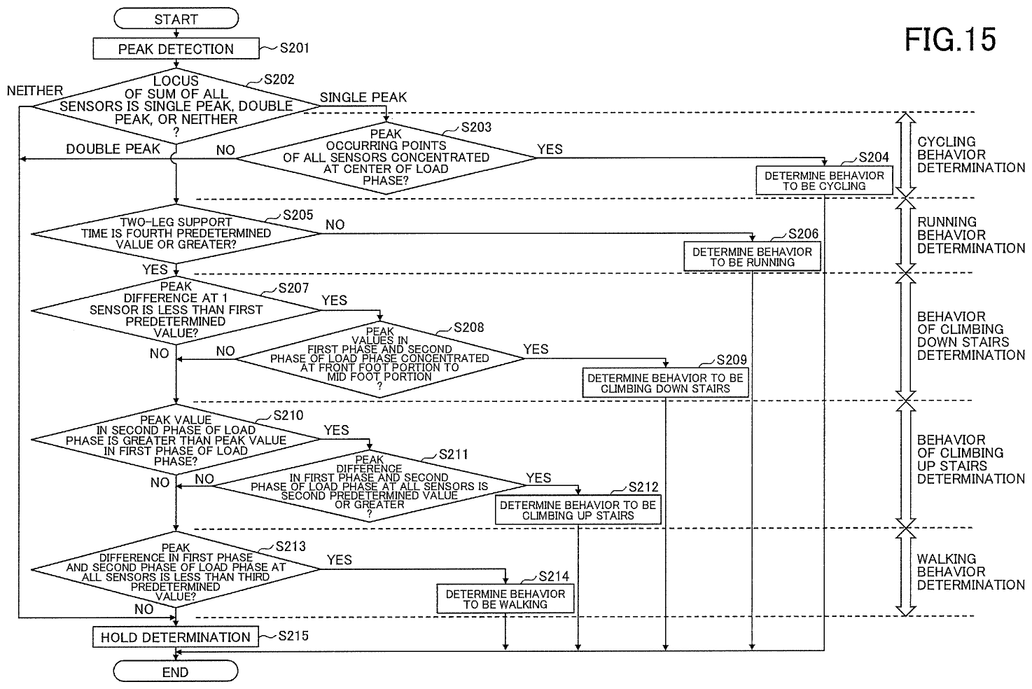

In step S116, the behavior determination device determines the behavior of the user based on the measurement data or the like. Details of the determination process will be described later in conjunction with FIG. 15.

<Examples of Measurement Data and 1 Load Phase Data>

FIG. 10A and FIG. 10B are diagrams illustrating an example of the measurement data. In FIG. 10A and FIG. 10B, an abscissa represents the time, and the ordinate represents the pressure value. Further, correspondence between the sensors No. 1 to No. 7 and the types of lines (solid line, one-dot chain line, or the like) used in FIG. 10A and FIG. 10B, is indicated on the right part of FIG. 10B for the sake of convenience.

For example, in step S111, the measurement data illustrated in FIG. 10A is acquired. Then, in step S112, the measurement data is analyzed to identify the 1 load phase, and when 1 load phase is extracted from the measurement data illustrated in FIG. 10A, the 1 load phase data illustrated in FIG. 10B, for example, can be generated.

Accordingly, a 1 load phase CYC illustrated in FIG. 10B becomes the stance time in the case where the behavior is walking.

<Example of Plantar Pressure Parameters>

FIG. 11 and FIG. 12 are diagrams illustrating an example of acquiring the plantar pressure parameters of 1 load phase. In FIG. 11 and FIG. 12, the abscissa represents a ratio in 1 load phase, and the ordinate represents the pressure value.

In step S113 or the like, the behavior determination device detects a point (hereinafter referred to as a “peak point”) where each waveform becomes a peak, and a local minimum point that occurs between a plurality of peak points. The peak point is the local maximum value or the maximum value of the waveform during a predetermined time.

For example, the behavior determination device determines the peak point or the local minimum point, by performing a differentiation or the like on the measurement data. The method of determining (or detecting) the peak point or the local minimum point may be other than the differentiation method, as long as the method can determine (or detect) the local maximum value or the local minimum value.

More particularly, when the maximum value or the like of each sensor in 1 load phase is detected, a value that is the maximum local maximum of each sensor in the first phase or the second phase of each sensor, such as a first peak point PM1 and a second peak point PM2 or the like, can be detected, and acquired as the plantar pressure parameter. In addition, when the maximum value of the sum of the pressure values of all of the sensors or the like is detected, peak points of the sum pressure such as PN1 through PN3, and the local minimum point such as PN4, can be detected by the pressure value, and acquired as the plantar pressure parameters. With regard to the sum of the pressure values, the two local maximum points PN1 and PN2, or the local minimum point PN4 amounting to the number of local maximum points minus 1, are detected in FIG. 11. With regard to the sum of the pressure values, 1 local maximum point PN3 is detected in FIG. 12. In FIG. 11 and FIG. 12, examples of the peak point and the local minimum point are illustrated by a black square symbol “ [symbol] ”.

Accordingly, when the process of step S113 is performed, the behavior determination device can acquire the plantar pressure parameters illustrated in FIG. 11 or FIG. 12, for example.

<Examples of Time Parameters>

In FIG. 11 and FIG. 12, the point in time (percentage) when the maximum local maximum of each sensor is detected in the first phase and the second phase of the load phase, is regarded as the “peak occurring point” of each sensor, and acquired as the time parameter.

More particularly, in FIG. 11, the first peak point PM1 is detected by one sensor at the point in time of 22 percent, and the second peak point PM2 is detected by a sensor, different from the sensor that detects the first peak point PM1, at the point in time of 48 percent. In FIG. 12, maximum local maximum points PM3 and PM4 are detected by one sensor during the first phase and the second phase of the 1 load phase of this sensor, at the point in time in a vicinity of 50 percent.

FIG. 13 is a diagram illustrating an example of the time parameter acquisition during the single-leg support phase. FIG. 13 illustrates the example of the time parameters for the case where the behavior is walking.

For example, a stance (or standing) time TS can be acquired by identifying the time when the pressure value that is a constant value or greater is measured. As illustrated in FIG. 13, the stance time TS is approximately equal to the time, from the time when the left foot makes contact with the ground to the time when the left foot thereafter separates from the ground, for example. In this example, the stance time TS becomes the 1 load phase.

In the following, as illustrated in FIG. 13, the time in which both the left and right feet make contact with the ground, may also be referred to as the “two-leg support phase”. On the other hand, the time in which only one of the left and right feet makes contact with the ground, may also be referred to as the “single-leg support phase”.

Accordingly, when process of step S114 is performed, the behavior determination device can acquire the time parameters illustrated in FIG. 13, for example.

<Example of Measurement Data to be Subjected to Behavior Determination Process>

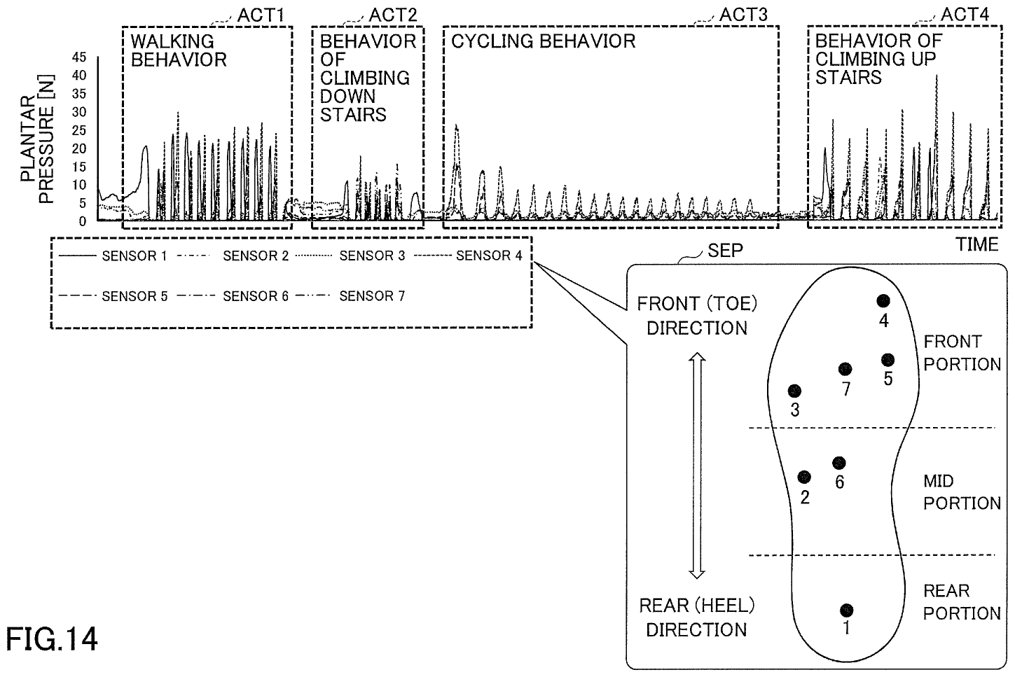

FIG. 14 is a diagram illustrating an example of the measurement data obtained by measuring four types of behaviors. An example will be described for a case where the measurement data illustrated in FIG. 14 are acquired.

In this example, the measurement data indicate the pressure values measured by each of the sensors when the user walks (hereinafter also referred to as performing a “walking behavior”) ACT1, a climbs down stairs (hereinafter also referred to as performing a “stair climbing down behavior”) ACT2, a cycles or rides a bicycle (hereinafter also referred to as performing a “cycling behavior”) ACT3, and climbs up stairs (hereinafter also referred to as performing a “stair climbing up behavior”) ACT4, as illustrated in FIG. 14.

In this example, the sensors are located at sensor positions SEP illustrated in FIG. 14.

The behavior determination device performs the overall process illustrated in FIG. 9 on the measurement data illustrated in FIG. 14. In step S116 of the overall process, the behavior determination device performs the following determination process, for example.

<Example of Behavior Determination Process>

FIG. 15 is a flow chart illustrating an example of a behavior determination process. The behavior determination process illustrated in FIG. 15 is performed based on the post-analysis data or the like, that may be acquired in advance by steps S113 and S114, and recorded in the server device or the like by step S115.

Further, in the example illustrated in FIG. 15, the behavior determination process is performed in the order of the cycling behavior determination, the running behavior determination, the stair climbing down behavior determination, the stair climbing up behavior, and the walking behavior determination, however, the order of each of the behavior determinations is not limited to the order illustrated in FIG. 15. For example, each behavior determination may be perfolmed separately.

<Example of Peak Detection> (Step S201)

In step S201, the behavior determination device detects a point (hereinafter, referred to as a “peak point”) where each wavefoim becomes a peak. More particularly, this step S201 utilizes the plantar pressure parameters recorded in the post-analysis data in step S115, to identify the peak point from the maximum local maximum average of each of the sensors in the first and the second phases of the single load phase. In addition, this step S201 utilizes the plantar pressure parameters identify one peak point or two peak points from the maximum local maximum average of the sum of pressure values of all of the sensors.

Further, when the maximum value or the like is calculated in advance from the measurement data, the behavior determination device may extract the peak point to be processed, from the maximum value or the like, even when the peak point does not depend from the post-analysis data.

<Example of Determining Whether Locus of Sum of all Sensors is Single Peak, Double Peak, or Neither> (Step S202)

In step S202, the behavior determination device determines whether or not the waveform of a sum total pressure value is a single peak, a double peak, or neither, using the parameters of the 1 load phase data of the sum of the pressure values of all of the sensors.

The single peak corresponds to a case where one peak point exists in the waveform of 1 load phase data. On the other hand, the double peak corresponds to a case where two peak points exist in the waveform of the 1 load phase, and there is a local minimum point between the two peak points. Accordingly, the behavior determination device can determine whether or not the a load phase data includes a single peak, a double peak, or neither, according to the number of peak points generated in the sum of the pressure values of all of the sensors within the 1 load phase data, and the existence (or non-existence) of the local minimum point. In this case, in order to clearly determine the double peak, the behavior determination device may make the determination based on an additional reference that, a difference between a lower pressure value of the two peaks, and the pressure value of the local minimum point is greater than or equal to a predetermined value.

When the behavior determination device determines that the load phase data has the single peak (“single peak” in step S202), the behavior determination device proceeds to step S203. On the other hand, when the behavior determination device determines that the 1 load phase data has the double peak (“double peak” in step S202), the behavior determination device proceeds to step S205. When the behavior determination device determines that the 1 load phase data has neither the single peak nor the double peak (“neither” in step S202), the behavior determination device proceeds to step S215.

<Example of Determining Whether Peak Occurring Point of all Sensors is Concentrated at Center of Load Phase> (Step S203).

In step S203, the behavior determination device determines whether or not the peak occurring points in the first phase and the peak occurring points in the second phase of all of the sensors are all concentrated at a center of the load phase. More particularly, the behavior determination device acquires the peak occurring points in the first phase and the second phase of all of the sensors, and determines that the peak occurring points of all of the sensors are concentrated at the center of the load phase, that is, the pressure in the 1 load phase is concentrated at a center time band, when all peak occurring points are 35% or more and 65% or less relative to 1 load phase. Further, when the peak occurring points of all of the sensors are concentrated at the center of the load phase (YES in step S203), the behavior determination device proceeds to step S204. On the other hand, when one of the peak occurring points is not 35% or more and 65% or less relative to 1 load phase (NO in step S203), the behavior determination device proceeds to step S215.

<Example of Determining Cycling Behavior> (Step S204)

In step S204, the behavior determination device determines that the user is cycling (that is, riding the bicycle).

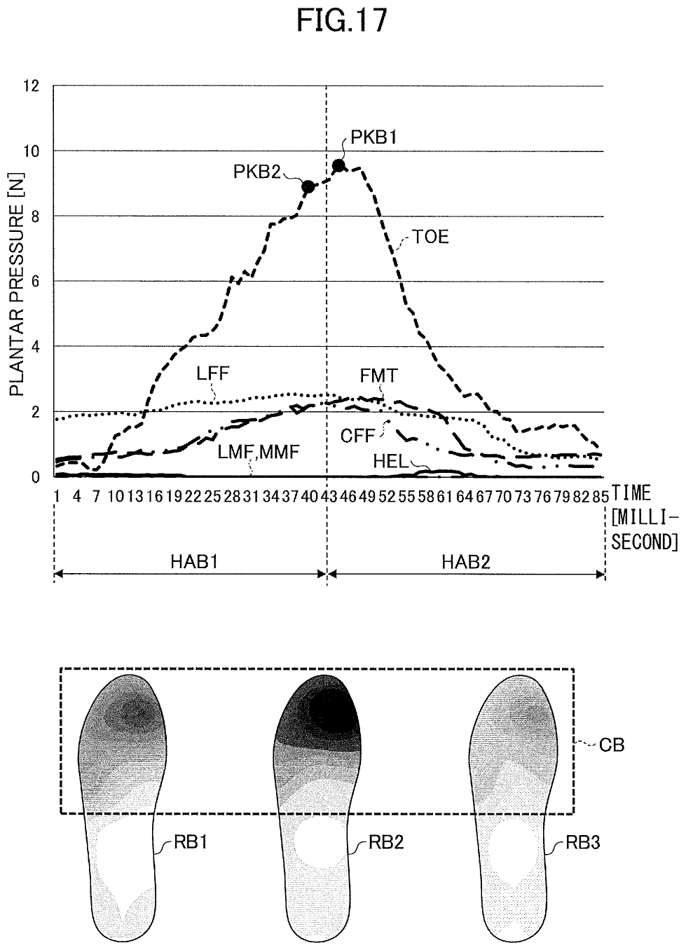

The cycling behavior determination method used in step S203, step S204, or the like will be described later in detail in conjunction with FIG. 17.

<Example of Determining Whether Two-Leg Support Time is Greater than or Equal to, or Less than Fourth Predetermined Value> (Step S205)

In step S205, the behavior determination device determines whether or not the two-leg support time is greater than or equal to a fourth predetermined value. More particularly, the behavior determination device first calculates the stance time of each foot, as illustrated in FIG. 13 or the like, and calculates the two-leg support time that becomes the “two-leg support phase” illustrated in FIG. 13. Next, the behavior determination device determines whether or not the two-leg support time, that is set in advance, is less than the fourth predetermined value, that is, whether or not the two-leg support time is short.

When the two-leg support time is greater than or equal to the fourth predetermined value in step S205 (YES in step S205), the behavior determination device proceeds to step S207. On the other hand, when the two-leg support time is less than the fourth predetermined value in step S205 (NO in step S205), the behavior determination device proceeds to step S206.

<Example of Determining Running Behavior> (Step S206)

In step S206, the behavior determination device determines that the user is running, that is, performing the running behavior.

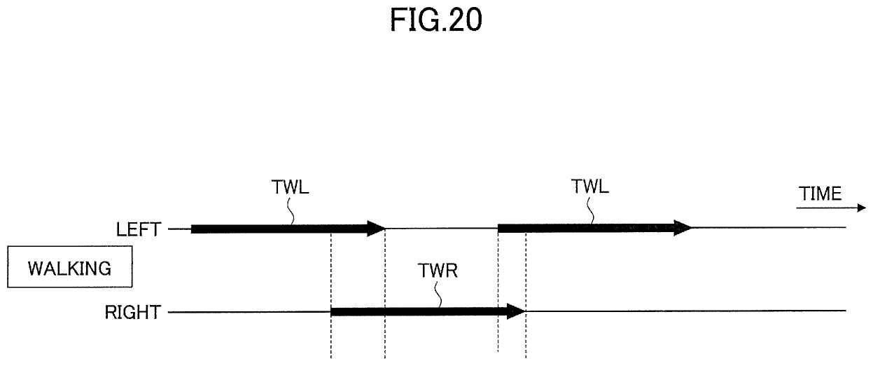

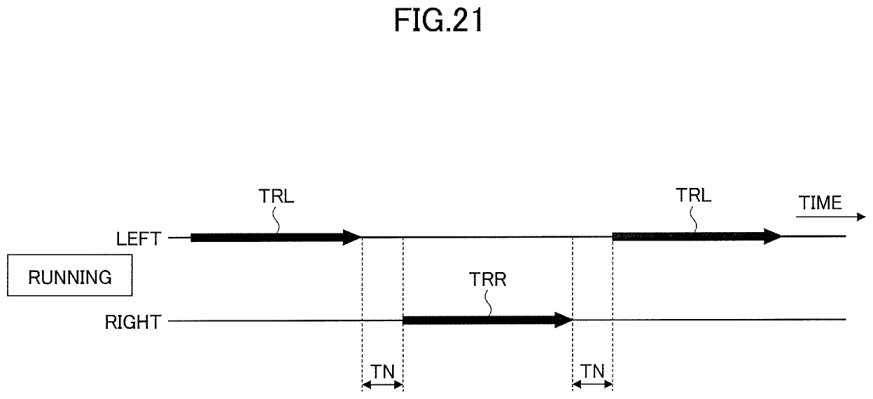

The running behavior determination method used in step S205, step S206, or the like will be described later in detail in conjunction with FIG. 20, FIG. 21, or the like.

<Example of Determining Whether Peak Difference in One Sensor is Less than First Predetermined Value> (Step S207).

In step S207, the behavior determination device determines whether or not the difference between the first and second phases of one sensor, that indicating a highest pressure value during the load phase, is less than a first predetermined value. More particularly, the behavior determination device compares the maximum local maximum averages of all of the sensors, and identifies one sensor indicating the highest value. With respect to this one sensor, the behavior determination device first calculates the difference between the values of the peak points in the first and second phases of the load phase, to calculate the peak difference. The behavior determination device then determines whether or not the peak difference is less than the first predetermined value that is set in advance, that is, whether or not the peak difference is a small value.

The predetermined value, such as the first predetermined value or the like, may be set to a different value for each person by taking the individual differences or the like into consideration.

When the peak difference in the one sensor is less than the first predetermined value (YES in step S207), the behavior determination device proceeds to step S208. On the other hand, when the peak difference in the one sensor is not less than the first predetermined value (NO in step S207), the behavior determination device proceeds to step S210.

<Example of Determining Whether Pressure or Force is Concentrated at Front Foot Portion to Mid Foot Portion> (Step S208).

In step S208, the behavior determination device determines whether pressure or force is concentrated at the front foot portion to the mid foot portion.

More particularly, the behavior determination device compares the maximum local maximum averages of each of the sensors in the first phase of the load phase, to check whether or not the sensor indicating the highest value and the sensor indicating the second highest value are the sensors provided at the front foot portion or the mid foot portion, and to make a similar check with respect to the second phase of the load phase, and to determine that the pressure or force is concentrated at the front foot portion or the mid foot portion when determination results of both the checking are in the affirmative (YES). In this case (YES in step S208), the behavior determination device proceeds to step S209. On the other hand, when the pressure or force is not concentrated at the front foot portion or the mid foot portion (NO in step S208), the behavior determination device proceeds to step S210.

<Example of Determining Stair Climbing Down Behavior> (Step S209)

In step S209, the behavior determination device determines that the user is climbing down the stairs, that is, performing the stair climbing down behavior.

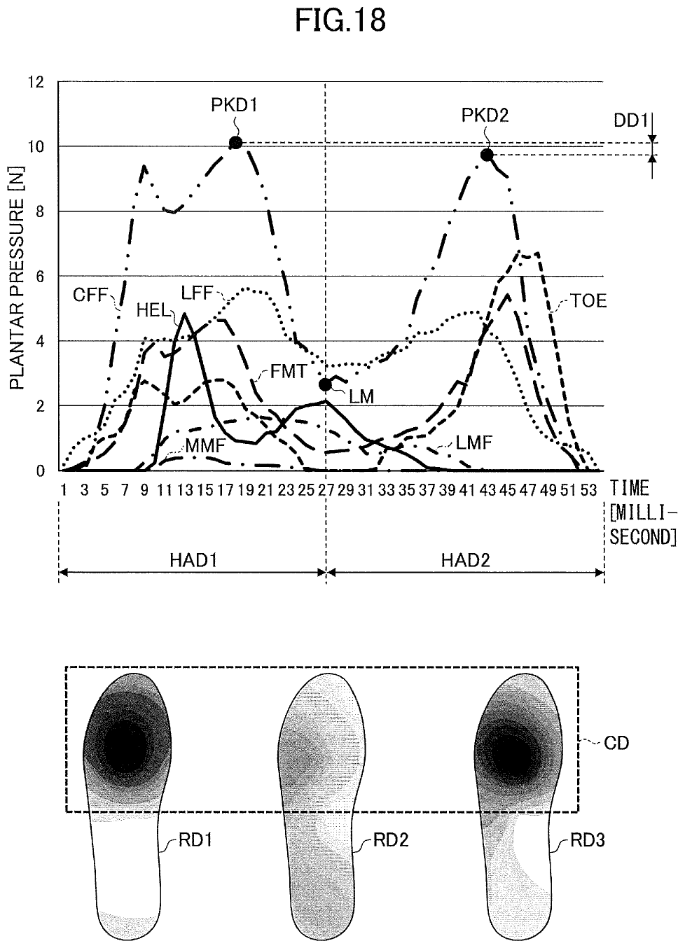

The stair climbing down behavior determination method used in step S207, step S208, step S209, or the like will be described later in detail in conjunction with FIG. 18.

<Example of Determining Whether Pressure During 1 Load Phase is Concentrated at Second Phase> (Step S210).

In step S210, the behavior determination device determines whether or not the pressure during 1 load phase is concentrated at the second load phase.

More particularly, the behavior determination device acquires the highest value (hereinafter referred to as the “peak value”) among the peak points (maximum local maximum averages) of each of the sensors in each of the first and second phases of 1 load phase, and determines whether or not the peak value of the second phase is higher than the peak value of the first phase.

When the peak value in the second phase is greater than the peak value in the first phase (YES in step S210), the behavior determination device proceeds to step S211. On the other hand, when the peak value in the second phase is smaller than or equal to the peak value in the first phase (NO in step S210), the behavior determination device proceeds to step S213.

<Example of Determining Whether Total Peak Difference of all Sensors is Greater than or Equal to Second Predetermined Value> (Step S211)

In step S211, the behavior determination device determines whether a total peak difference of all of the sensors is greater than or equal to a second predetermined value. More particularly, the behavior determination device first calculates the difference between the peak values from the peak points in the first and second phases of the 1 load phase, to obtain the peak difference (hereinafter referred to as the “total peak difference”). The behavior determination device then determines whether or not the total peak difference is greater than or equal to the second predetermined value that is set in advance, that is, whether or not the total peak difference is a large value.

When the total peak difference is greater than or equal to the second predetelmined value (YES in step S211), the behavior determination device proceeds to step S212. On the other hand, when the total peak difference is not greater than or equal to the second predetermined value (NO in step S211), the behavior determination device proceeds to step S213.

<Example of Determining Stair Climbing Up Behavior> (Step S212)

In step S212, the behavior determination device determines whether or not the user is climbing up the stairs, that is, performing the stair climbing up behavior.

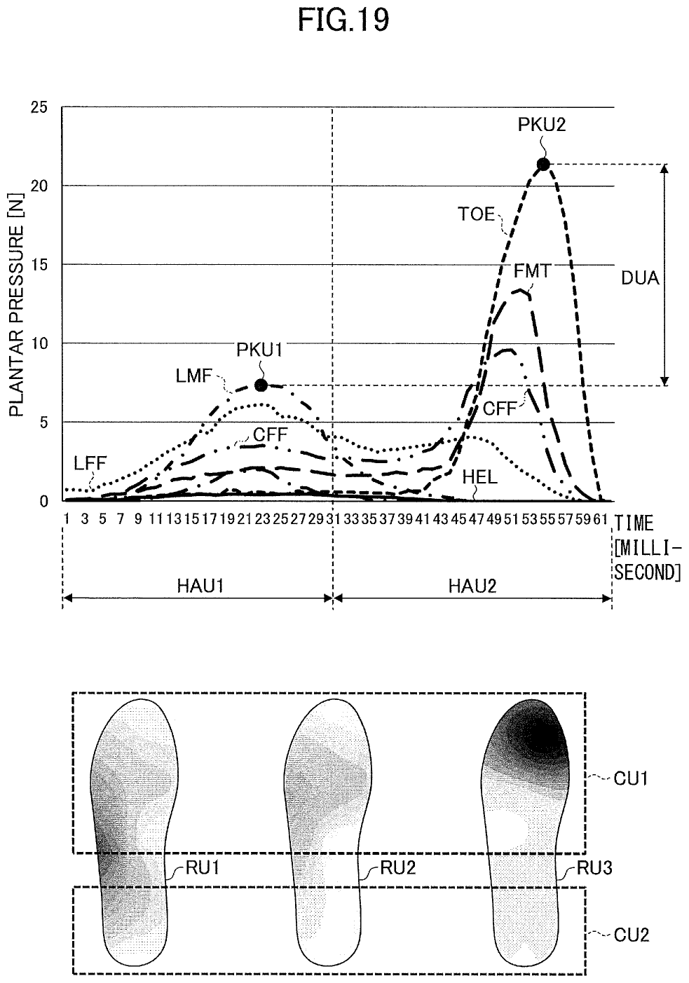

The stair climbing up behavior determination method used in step S210, step S211, step S212, or the like will be described later in detail in conjunction with FIG. 19.

<Example of Determining Whether Total Peak Difference is Less than Third Predetermined Value> (Step S213)

In step S213, the behavior determination device determines whether or not the total peak difference is less than a third predetermined value. More particularly, the behavior determination device acquires the total peak difference. Then, the behavior determination device determines whether or not the total peak difference is less than the third predetermined value that is set in advance, that is, whether or not the total peak difference is a small value. When the total peak difference is less than the third predetermined value (YES in step S213), the behavior determination device proceeds to step S214. On the other hand, when the total peak difference is not less than the third predetermined value (NO in step S213), the behavior determination device proceeds to step S215.

<Example of Determining Walking Behavior> (Step S214)

In step S214, the behavior determination device determines whether or not the user is walking, that is, performing the walking behavior.

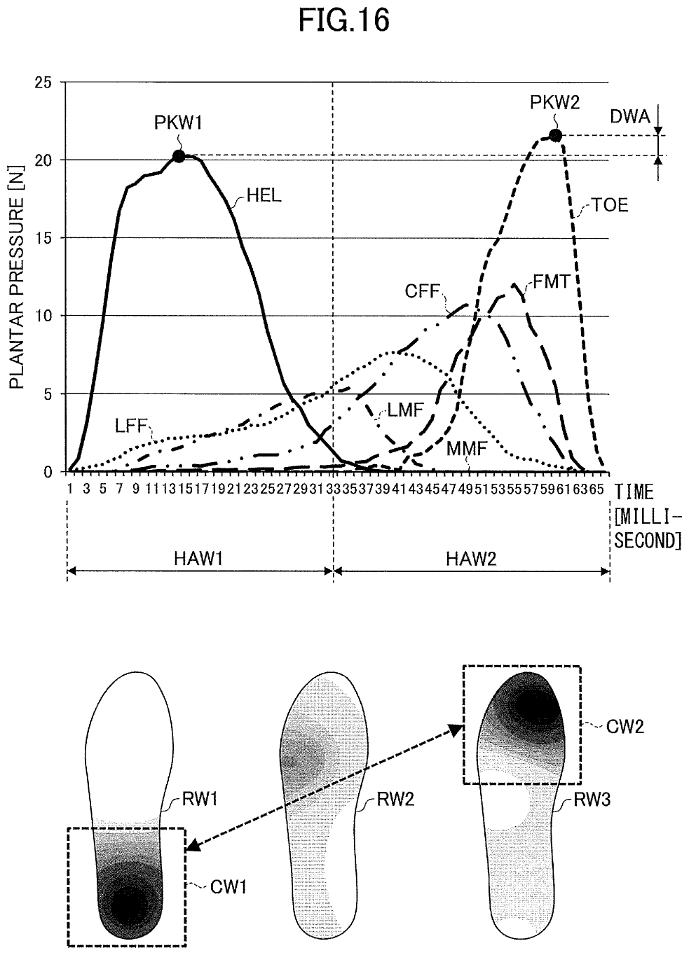

The walking behavior determination method used in step S213, step S214, or the like will be described later in detail in conjunction with FIG. 16 or the like.

<Example of Holding Determination> (Step S215)

In step S215, the behavior determination device holds (or reserves) the determination of the behavior during a time band in which a particular behavior determination is not reached, and ends the behavior determination process.

The behavior determination process described above may be performed for every a load phase, for example. The behavior determination process is not limited to being performed for every 1 load phase, and may be performed at predetermined intervals, such as for every period, every interval, or the like that is set in advance.

<Example of Determining Walking Behavior>

FIG. 16 is a diagram illustrating an example of the 1 load phase data measured from the walking behavior.

When the user is performing the walking behavior ACT1, 1 load phase data illustrated in FIG. 16, for example, is generated. First, as illustrated in FIG. 16, based on the 1 load phase data during the walking behavior ACT1, two peak points are detected in step S201, as illustrated by an eleventh peak point PKW1 and a twelfth peak point PKW2 in the first phase and the second phase, respectively, for all of the sensors. As illustrated in FIG. 16, the sensor of the eleventh peak point PKW1 and the sensor of the twelfth peak point PKW2 are different sensors.

Accordingly, in the case of the walking behavior ACT1, the 1 load phase data has the double peak.

In this case, the 1 load phase data during the walking behavior ACT1 has a small first total peak difference DWA, which is the difference between the eleventh peak point PKW1 and the twelfth peak point PKW2. Accordingly, in the case of the walking behavior ACT1, the first total peak difference DWA has a value less than the third predetermined value. For this reason, the determination result in step S213 becomes YES.

In addition, a distribution of the pressure values is preferably taken into consideration in the determination of the walking behavior, as described in the following.

First, when 1 load phase is equally divided into two phases, namely, a stance first phase HAW1 and a stance second phase HAW2, a pressure distribution such as that of an eleventh measurement result RW1 is measured in the stance first phase HAW1, while a pressure distribution such as that of a thirteenth measurement result RW3 is measured in the stance second phase HAW2. As illustrated in FIG. 16, the pressure distribution such as that of a twelfth measurement result RW2 is measured at an intermediate point in time between the stance first phase HAW1 and the stance second phase HAW2.

The eleventh measurement result RW1 is an example with a first distribution. As illustrated in FIG. 16, the eleventh measurement result RW1 has a distribution (hereinafter referred to as a “first concentrated distribution CW1”) in which the pressure is concentrated at the rear foot portion, such as the heel or the like.

The thirteenth measurement result RW3 is an example with a second distribution. As illustrated in FIG. 16, the thirteenth measurement result RW3 has a distribution (hereinafter referred to as a “second concentrated distribution CW2”) in which the pressure is concentrated at the front foot portion, such as the toe or the like.

In the case of the sensor positions SEP illustrated in FIG. 14, the pressure or force generated at the rear foot portion can be measured because the No. 1 sensors or the like are provided. Accordingly, the behavior determination device can determine whether or not the first concentrated distribution CW1 is obtained in the stance first phase HAW1, by determining whether or not the eleventh peak point PKW1 is positioned at the rear foot portion.

Similarly, in the case of the sensor positions SEP illustrated in FIG. 14, the pressure or force generated at the front foot portion can be measured because the No. 4 sensors or the like are provided. Accordingly, the behavior determination device can determine whether or not the second concentrated distribution CW2 is obtained in the stance second phase HAW2, by determining whether or not the twelfth peak point PKW2 is positioned at front foot portion.

As described above, in the case of the walking behavior, the first concentrated distribution CW1 is generated in the stance first phase HAW1, while the second concentrated distribution CW2 is generated in the stance second phase HAW2. In other words, the first distribution and the second distribution are generated periodically in the case of the walking behavior.

Accordingly, the behavior determination device can determine walking behavior by determining whether or not the first distribution and the second distribution are generated periodically. By determining the periodically generated first and second distributions, the behavior determination device can more accurately determine the walking behavior ACT1.

<Example of Determining Cycling Behavior>

FIG. 17 is a diagram illustrating an example of the 1 load phase data measured from the cycling behavior.

When the user is riding the bicycle, that is, performing the cycling behavior ACT3, 1 load phase data illustrated in FIG. 17, for example, is generated. First, as illustrated in FIG. 17, based on the 1 load phase data during the cycling behavior ACT3, two peak points are detected in step S201, as illustrated by a second peak point PKB1 and a third peak point PKB2 in the first phase and the second phase, respectively, for all of the sensors. In this case, there is no noticeable local minimum point exists between the second peak point PKB1 and the third peak point PKB2. In addition, the peak occurring point of each peak point is 35% or more and less than 50% relative to 1 load phase for the second peak point PKB1, and 50% or more and 65% or less relative to 1 load phase for the third peak point PKB2. In other words, both the peak point in the first phase of the load phase, and the peak point in the second phase of the load phase are concentrated near the center of the load phase. Accordingly, in the case of the cycling behavior ACT3, the 1 load phase data has the single peak. For this reason, the determination result in step S203 becomes YES.

In addition, a distribution of the pressure values is preferably taken into consideration in the determination of the cycling behavior, as described in the following.

First, similar to FIG. 16, when the 1 load phase is equally divided into a behavior first phase HAB1 and a behavior second phase HAB2, a pressure distribution such as that of a twenty-first measurement result RB1 is measured in the behavior first phase HAB1, while a pressure distribution such as that of a twenty-third measurement result RB3 is measured in the behavior second phase HAB2. As illustrated in FIG. 17, the pressure distribution such as that of a twenty-second measurement result RB2 is measured at the intermediate point in time between the behavior first phase HAB1 and the behavior second phase HAB2.

As indicated by a twenty-first measurement result RB1, a twenty-second measurement result RB2, and a twenty-third measurement result RB3, in the cycling behavior, the pressure or force is concentrated at predetermined points of the foot portion in many cases. In the example illustrated in FIG. 17, the pressure or force is concentrated at the front foot portion to the mid foot portion, as indicated by a third distribution CB. The predetermined points where the pressure or force is concentrated differ depending on the person. In other words, the predetermined points where the pressure or force is concentrated are not limited to the front foot portion to the mid foot portion, as in the case of the third distribution CB.

Accordingly, the behavior determination device determines whether or not the pressure or force is concentrated at the predetermined points of the foot portion, as in the case of the third distribution CB, to determine the cycling behavior. By further performing such a determination, the behavior determination device can more accurately determine the cycling behavior ACT3.

<Example of Determining Stair Climbing Down Behavior>

FIG. 18 is a diagram illustrating an example of the 1 load phase data measured from the stair climbing down behavior.

When the user is performing a stair climbing down behavior ACT2, a load phase data illustrated in FIG. 18, for example, is generated. First, as illustrated in FIG. 18, based on the 1 load phase data during the stair climbing down behavior ACT2, two peak points are detected in step S201, as illustrated by a thirty-first peak point PKD1 and a thirty-second peak point PKD2, for all of the sensors. As illustrated in FIG. 18, the sensor of the thirty-first peak point PKD1 and the sensor of the thirty-second peak point PKD2 are the same, unlike the case illustrated in FIG. 16. The sensors used for the determination are sensors that indicate the maximum pressure value.

Hence, even in the case where two peak points are detected by one sensor, the 1 load phase data has the double peak.

In addition, the 1 load phase data during the stair climbing down behavior ACT2 has a first peak difference DD1 of one sensor, that is the difference between the thirty-first peak point PKD1 and the thirty-second peak point PKD2, and this first peak difference DD1 is a small value. Accordingly, the first peak difference DD1 is less than the first predetermined value during the stair climbing down behavior ACT2. For this reason, in step S207, it is determined that the first peak difference DD1 is less than the first predetermined value.

In addition, the distribution of the pressure values is taken into consideration as follows when determining the stair climbing down behavior.

First, when the 1 load phase is equally divided into two phases, namely, a stance first phase HAD1 and a stance second phase HAD2, similar to FIG. 16, a pressure distribution such as that of a thirty-first measurement result RD1 is measured in the stance first phase HAD1, while a pressure distribution such as that of a thirty third measurement result RD3 is measured in the stance second phase HAD2. As illustrated in FIG. 18, the pressure distribution such as that of a thirty-second measurement result RD2 is measured at an intermediate point in time between the stance first phase HAD1 and the stance second phase HAD2.

As indicated by the thirty-first measurement result RD1, the thirty-second measurement result RD2, and the thirty-third measurement result RD3, during the stair climbing down behavior, the pressure or force is concentrated at the front foot portion or the mid foot portion in many cases. Whether or not the pressure or force is concentrated at the front foot portion or the mid foot portion may be determined, by determining whether or not the sensor indicating the peak point in the first phase of the load phase is positioned at the front foot portion or the mid foot portion, and similarly determining whether or not the sensor indicating the peak point in the second phase of the load phase is positioned at the front foot portion or the mid foot portion. A further determination may be added to determine whether or not the sensor indicating the second largest maximum local maximum average in the first phase of the load phase is positioned at the front foot portion or the mid foot portion, and whether or not the sensor indicating the second largest maximum local maximum average in the second phase of the load phase is positioned at the front foot portion or the mid foot portion.

In the example illustrated in FIG. 18, the pressure or force is concentrated at the front foot portion to the mid foot portion, as indicated by a fourth distribution CD. As illustrated in FIG. 18, in the 1 load phase data, the pressure values indicated by the No. 7 sensor CFF, the No. 4 sensor TOE, and the No. 5 sensor FMT that are positioned to measure the pressure at the front foot portion, among the sensor positions illustrated in FIG. 7, are high. In other words, this is an example where the pressure is concentrated at the front foot portion.

Accordingly, the behavior judgement device determines whether or not the pressure or force is concentrated at the front foot portion or the mid foot portion, as in the fourth distribution CD, to determine the stair climbing down behavior. For this reason, the determination result in step S208 becomes YES.

Further, it is more desirable for the behavior determination device to consider whether or not a local minimum point LM exists between the peak point in the first phase of the load phase and the peak point in the second phase of the load phase, with respect to one sensor indicating the peak point. As illustrated in FIG. 18, during the stair climbing down behavior, the local minimum point LM exists in many cases because one sensor indicates the double peak. The local minimum point LM can be detected by differentiation or the like, for example. Accordingly, the behavior determination device detects the local minimum point LM, to determine the stair climbing down behavior. When such a determination is further performed, the behavior determination device can more accurately determine the stair climbing down behavior ACT2.

<Example of Determining Stair Climbing Up Behavior>

FIG. 19 is a diagram illustrating an example of the 1 load phase data measured from the stair climbing up behavior.

When the user is performing the stair climbing up behavior ACT4, 1 load phase data illustrated in FIG. 19, for example, is generated. First, as illustrated in FIG. 19, based on the 1 load phase data during the stair climbing up behavior ACT4, two peak points are detected in step S201, as illustrated by a forty-first peak point PKU1 and a forty-second peak point PKU2, for all of the sensors. As illustrated in FIG. 19, the sensor of the forty-first peak point PKU1 and the sensor of the forty-second peak point PKU2 are different sensors, unlike the case illustrated in FIG. 18.

Accordingly, in the case of the stair climbing up behavior ACT4, the 1 load phase data has the double peak. In this case, the peak value in the second phase of the load phase always becomes larger than the peak value in the first phase of the load phase. In the example illustrated in FIG. 19, the forty-second peak point PKU2 is larger than the forty-first peak point PKU1. For this reason, the determination result in step S210 becomes YES.

In addition, in the 1 load phase data during the stair climbing up behavior ACT4, a second total peak difference DUA, that is the difference between the forty-first peak point PKU1 and the forty-second peak point PKU2, is a large value. In the example illustrated in FIG. 19, the second total peak difference DUA is approximately three times larger than the first total peak difference DWA illustrated in FIG. 16. The difference between the stair climbing up behavior and the walking behavior differ depending on the person. Accordingly, the second full peak difference DUA is greater than or equal to the second predetermined value during the stair climbing up behavior ACT4. For this reason, in step S211, it is deteiinined that the second total peak difference DUA is greater than or equal to the second predetermined value.

The peak points used to calculate the second full peak difference DUA is a combination of the peak point (hereinafter referred to as the “first peak point”) occurring in the first half of the 1 load phase, and the peak point (hereinafter referred to as the “second peak point”) occurring in the second half of the 1 load phase. In this example, the first peak point is the forty-first peak point PKU1, and the second peak point is the forty-second peak point PKU2.

As illustrated in FIG. 19, the 1 load phase is first equally divided into two phases, namely, a stance first phase HAU1 and a stance second phase HAU2, similar to FIG. 16. In this example, the first half of the 1 load phase is the stance first phase HAU1, and the second half of the 1 load phase is the stance second phase HAU2. Accordingly, the second full peak difference DUA is the value of the calculated difference between the peak point detected in the stance first phase HAU1 and the peak point detected in the stance second phase HAU2.

In addition, the distribution of pressure values is desirably taken into consideration as follows when determining the stair climbing up behavior. For example, in the stance first phase HAU1, a pressure distribution such as that of a forty-first measurement result RU1 is measured, while in the stance second phase HAU2, a pressure distribution such as that of a forty-third measurement result RU3 is measured. As illustrated in FIG. 19, the pressure distribution such as that of a forty-second measurement result RU2 is measured at an intermediate point in time between the stance first phase HAU1 and the stance second phase HAU2.

As indicated by the forty-first measurement result RU1, the forty-second measurement result RU2, and the forty-third measurement result RU3, during the stair climbing up behavior, the pressure or force is concentrated at the front foot portion or the mid foot portion in many cases. In the example illustrated in FIG. 19, the pressure or force is concentrated at the front foot portion to the mid foot portion, as indicated by a fifty-first distribution CU1.

Moreover, during the stair climbing up behavior, the pressure or force is not generated at the rear foot portion in many cases. In the example illustrated in FIG. 19, the pressure or force is not generated at the rear foot portion, as indicated by a fifty-second distribution CU2.

As illustrated in FIG. 19, in the 1 load phase data, the pressure values indicated by the No. 7 sensor CFF, the No. 4 sensor TOE, and the No. 5 sensor FMT that are positioned to measure the pressure at the front foot portion, among the sensor positions illustrated in FIG. 7, are high. In other words, this is an example where the pressure is concentrated at the front foot portion.

On the other hand, as illustrated in FIG. 19, in the 1 load phase data, the pressure value indicated by the No. 1 sensor HEL that is positioned to measure the pressure at the rear foot portion, among the sensor positions illustrated in FIG. 7, is low. In other words, this is an example where the pressure is not generated at the rear foot portion.

Accordingly, the behavior judgement device determines whether or not the pressure or force is concentrated at the front foot portion or the mid foot portion, as in the fifty-first distribution CUL and whether or not the pressure or force is concentrated at the rear foot portion, as in the fifty-second distribution CU2, to determine the stair climbing up behavior. By further performing such a detemination, the behavior determination device can more accurately determine the stair climbing up behavior ACT4.

<Example of Determining Running Behavior>

FIG. 20 is a diagram (part 1) illustrating an example of determining the running behavior, and FIG. 21 is a diagram (part 2) illustrating the example of determining the running behavior.

In order to determine whether or not the behavior is the running behavior, it is desirable for the behavior determination device to use the stance time of each foot.

First, in comparison with control, during the walking behavior, the stance time of the left foot is the eleventh stance time TWL or the like illustrated in FIG. 20, for example. On the other hand, during the walking behavior, the stance time of the right foot is the twelfth stance time TWR or the like illustrated in FIG. 20, for example.

Similarly, during the running behavior, the stance time of the left foot is the twenty-first standing time TRL or the like illustrated in FIG. 21, for example. On the other hand, during the running behavior, the stance time of the right foot is the twenty-second stance time TRR or the like illustrated in FIG. 21.

The stance time, such as the eleventh stance time TWL, the twelfth stance time TWR, the twenty-first stance time TRL, and the twenty-second stance time TRR, is a value calculated using the time parameters illustrated in FIG. 6, for example.

During the running behavior, an overlap in the stance times of the feet, that is, the “two-leg support time” illustrated in FIG. 6, is shorter than that during the walking behavior. In the example illustrated in FIG. 21, a blank period TN exists between the twenty-first stance time TRL and the twenty-second stance time TRR, and the two-leg support time in which the user stands on both feet is “0.” On the other hand, the example illustrated in FIG. 20 does not include the blank period TN. Accordingly, in the case of the running behavior, it is determined in step S205 that the two-leg support time is not greater than or equal to the fourth predetermined time that is set in advance.

In addition, when determining the running behavior, it is desirable to consider that the twenty-first stance time TRL and the twenty-second stance time TRR are shorter than the eleventh stance time TWL and the twelfth stance time TWR. In other words, during the running behavior, the stance time of each foot, that is, the time during which each foot makes contact with the ground, is shorter than that during the walking behavior in many cases. Accordingly, after determining the walking behavior in advance, the behavior determination device may compare the time parameter such as the stance time or the like of the walking behavior with that during the running behavior, and determine that the behavior is the running behavior when the stance time is shorter than that during the walking behavior. When such a determination is further performed, the behavior determination device can more accurately determine the running behavior ACTT.

<Results of Experiments>

The following results were obtained by performing the measurements on a plurality of persons.

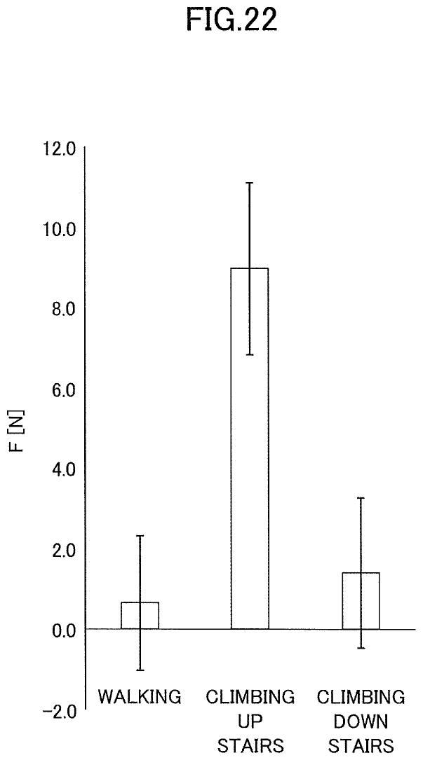

FIG. 22 illustrates an example of the peak difference between the first and second phases of the 1 load phase.

As a result of the experiments, there was a peak difference between the first phase and the second phase of the measurement data of each of the walking behavior, the stair climbing up behavior, and the stair climbing down behavior. As illustrated in FIG. 22, the peak difference was “9.1±2.11 [N]” for the stair climbing up behavior, which is larger than those of other behaviors. This indicates the appropriateness of using the first total peak difference DWA in step S213, the first peak difference DD1 in step S207, and the second total peak difference DUA in step S211 for the determination.

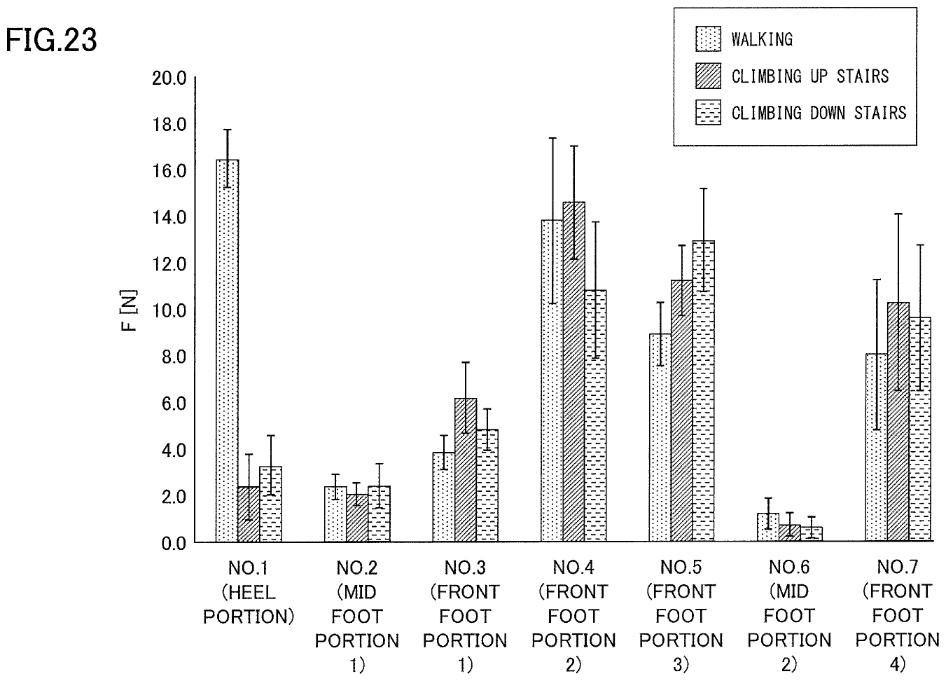

FIG. 23 is a diagram illustrating an example of the peak value of each sensor for each behavior.

As illustrated in FIG. 23, the pressure value at the heel portion was “16.4±1.26 [N]” during the walking behavior, which is larger than those at other portions.

### SUMMARY

As described above, when the peak points are used, since the values of the peak occurring point, the peak difference, and the total peak difference can be calculated in the behavior determination, the behavior determination device can accurately determine the behavior of the user. In addition, by performing the above described behavior determination process, the behavior determination device can also determine the behaviors, such as the stair climbing up behavior, the stair climbing down behavior, or the like, which could not be detelmined by conventional methods.

### OTHER EMBODIMENTS

The behavior determination process may perform the following process, for example.

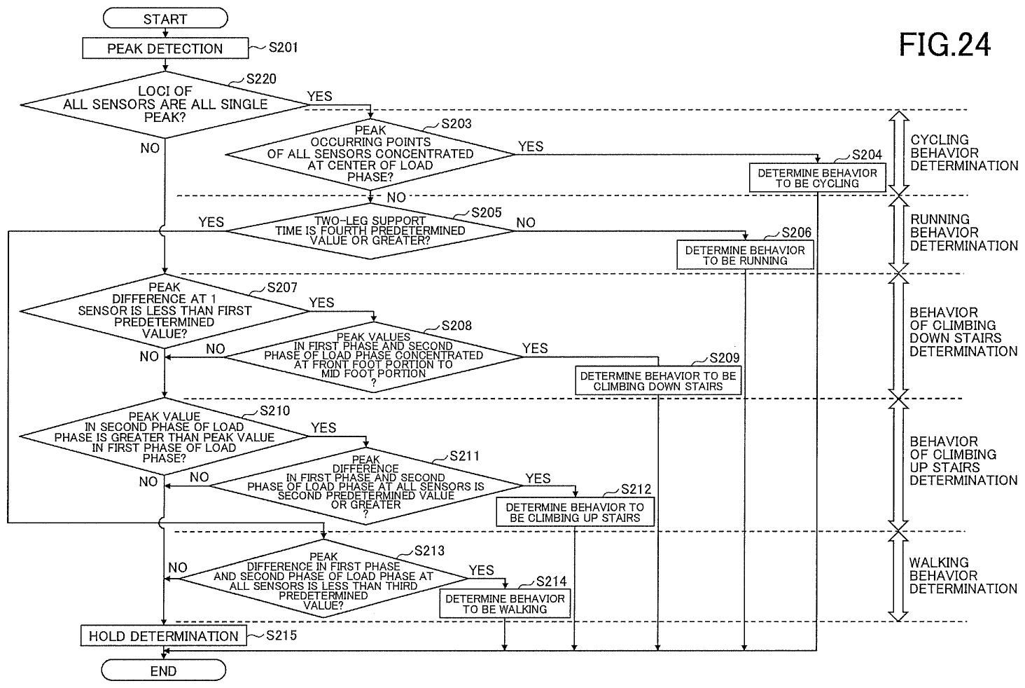

FIG. 24 is a flow chart illustrating a modification of the behavior determination process. Compared to the process illustrated in FIG. 15, the difference in FIG. 24 is that step S202 is replaced by step S220. In the following, steps that are the same as those steps illustrated in FIG. 15 are designated by the same reference numerals, and the description thereof will be omitted.

<Example of Determining Whether Locus of all Sensors is Single Peak> (Step S220)

In step S220, the behavior determination device determines whether or not loci of all of the sensors are the single peak. When the behavior determination device determines that the loci of all the sensors are the single peak (YES in step S220), the behavior determination device proceeds to step S203. On the other hand, when the behavior determination device determines that the loci of all of the sensors include a locus that is not the single peak (NO in step S220), the behavior determination device proceeds to step S207.

For example, even when the above described process is performed, the same result as that illustrated in FIG. 15 or the like can be obtained.

In addition, an analysis may be performed by combining the result of the behavior determination with life log data. For example, an intensity of movement may be analyzed, or a relationship between a geographic location and the behavior may be analyzed. By performing such an analysis, the behavior determination device can perform a more detailed monitoring of biometric information of the user.

In the description given heretofore, the pressure is mainly measured as an example, however, the force may be measured using a force sensor. In addition, in a state where an area in which the force is to be measured is known in advance, the force may be measured, and the measured force may be divided by the area, to calculate the pressure or the like.

The behavior determination system 100 is not limited to the system configuration illustrated in FIG. 1. In other words, the behavior determination system 100 may further include an information processing device other than that illustrated in the FIG. 1. On the other hand, the behavior determination system 100 may be formed by one or a plurality of information processing devices, and may be formed by a number of information processing devices smaller than the number of information processing devices illustrated in FIG. 1.

Each device does not necessarily have to be formed by one device. In other words, each device may be formed by a plurality of devices. For example, each device in the behavior determination system 100 may perform each process by a distributed processing, a parallel processing, or a redundant processing executed by the plurality of devices.

All or a portion of each process according to the embodiments and modifications may be described in a low-level language, such as an assembler or the like, or a high-level language, such as an object-oriented language or the like, and may be performed by executing a program that causes the computer to perform a behavior determination method. In other words, the program may be a computer program for causing the computer, such as the information processing system or the like including the information processing device or the plurality of information processing devices, to execute each process.

Accordingly, when the behavior determination method is executed based on the program, the arithmetic unit and the control unit of the computer perform calculations and control based on the program for executing each process. The storage device of the computer stores the data used for the processing, based on the program, in order to execute each process.

The program may be stored and distributed on a non-transitory computer-readable storage medium. The non-transitory computer-readable storage medium includes a medium such as an auxiliary storage device, a magnetic tape, a flash memory, an optical disk, a magneto-optical disk, a magnetic disk, or the like. In addition, the program may be distributed over a telecommunication line.

Although the preferred embodiments of the present invention are described above in detail, the present invention is not limited to the embodiments described above, and various modifications, variations, and substitutions may be made within the scope of the present invention.

According to each of the embodiments and modification described above, it is possible to accurately determine the behavior of the user.

Although embodiments are described in detail above, the present invention is not limited to particular embodiments, and various variations, modifications, and substitutions may be made without departing from the scope of the present invention.

What is claimed is:

1. A behavior determination device comprising:
a measurement data receiving device configured to acquires measurement data indicating a pressure or a force measured by one or a plurality of sensors provided on a sole surface of a user's foot;
a data analyzing device configured to analyze the measurement data, to identify one load phase in which the user makes one step, and calculate a plantar pressure parameter and a time parameter for every one load phase; and
a behavior determining device configured to detect a peak point where a maximum local maximum is obtained for every predetermined time, based on the plantar pressure parameter and the time parameter, and determine a behavior of the user based on the peak point.

2. The behavior determination device as claimed in claim 1, wherein
the plurality of sensors include at least one sensor provided at a rear foot portion on the sole surface, and at least one sensor provided at a front foot portion on is installed at least one at the rear portion and the front portion on the sole surface, and
the behavior determining device compares the measurement data of all of the sensors, and determines the user's behavior as a walking behavior when a total peak difference, that is a difference between a peak value in a first phase of the one load phase and a peak value of a second phase of the one load phase, is less than a third predetermined value.

3. The behavior determination device as claimed in claim 1, wherein the behavior determining device determines the user's behavior as a cycling behavior, based on the peak point detected in the one load phase, when one peak point is detected during a predetermined time.

4. The behavior determination device as claimed in claim 1, wherein
the plurality of sensors include at least one sensor provided at each of a front foot portion, a mid foot portion, and a rear foot portion on the sole surface, and
the behavior determining device determines, based on the measurement data of each of the at least one sensor, the user's behavior as a stair climbing down behavior when a peak difference, that is a difference between a peak value in a first phase of the one load phase and a peak value of a second phase of the one load phase, is less than a first predetermined value, and the pressure or the force is concentrated at the front foot portion or the mid foot portion.

5. The behavior determination device as claimed in claim 4, wherein the behavior determining device further detects a local minimum point of the one sensor having the peak point, and determines the user's behavior as the stair climbing down behavior when the local minimum point is detected.

6. The behavior determination device as claimed in claim 1, wherein
the plurality of sensors are provided on the sole surface, and
the behavior detemining device compares the measurement data of all of the sensors, and determines the user's behavior as a stair climbing up behavior when a total peak difference between a peak value in a first phase of the one load phase and a peak value in a second phase of the one load phase is greater than or equal to a second predetermined value, and the peak value in the second phase of the one load phase is larger than the peak value of the first phase of the one load phase.

7. The behavior determination device as claimed in claim 1, wherein
the data analyzing device
calculates, as the time parameter, a stance time in which the user stands on each foot, for each foot, and
calculates a two-leg support time in which the user stand on both feet, based on the stance time calculated for each foot, and
the behavior determining device determines the user's behavior as a running behavior when the two-leg support time is less than the fourth predetermined value.

8. A behavior determination system comprising:
one or a plurality of information processing devices,
wherein the one or the plurality of information processing devices include a processor that performs a process including
acquiring measurement data indicating a pressure or a force measured by one or a plurality of sensors provided on a sole surface of a user's foot;
analyzing the measurement data, to identify one load phase in which the user makes one step, and calculating a plantar pressure parameter and a time parameter for every one load phase; and
detecting a peak point where a maximum local maximum is obtained for every predetermined time, based on the plantar pressure parameter and the time parameter, and determining a behavior of the user based on the peak point.

9. The behavior determination system as claimed in claim 8, wherein
the plurality of sensors include at least one sensor provided at a rear foot portion on the sole surface, and at least one sensor provided at a front foot portion on is installed at least one at the rear portion and the front portion on the sole surface, and
the detecting the peak point includes comparing the measurement data of all of the sensors, and determining the user's behavior as a walking behavior when a total peak difference, that is a difference between a peak value in a first phase of the one load phase and a peak value of a second phase of the one load phase, is less than a third predetermined value.

10. The behavior determination system as claimed in claim 8, wherein the detecting the peak point includes determining the user's behavior as a cycling behavior, based on the peak point detected in the one load phase, when one peak point is detected during a predetermined time.

11. The behavior determination system as claimed in claim 8, wherein
the plurality of sensors include at least one sensor provided at each of a front foot portion, a mid foot portion, and a rear foot portion on the sole surface, and
the detecting the peak point includes determining, based on the measurement data of each of the at least one sensor, the user's behavior as a stair climbing down behavior when a peak difference, that is a difference between a peak value in a first phase of the one load phase and a peak value of a second phase of the one load phase, is less than a first predetermined value, and the pressure or the force is concentrated at the front foot portion or the mid foot portion.

12. The behavior determination system as claimed in claim 11, wherein the detecting the peak point further includes detecting a local minimum point of the one sensor having the peak point, and determining the user's behavior as the stair climbing down behavior when the local minimum point is detected.

13. The behavior determination system as claimed in claim 8, wherein
the plurality of sensors are provided on the sole surface, and
the detecting the peak point includes comparing the measurement data of all of the sensors, and determining the user's behavior as a stair climbing up behavior when a total peak difference between a peak value in a first phase of the one load phase and a peak value in a second phase of the one load phase is greater than or equal to a second predetermined value, and the peak value in the second phase of the one load phase is larger than the peak value of the first phase of the one load phase.

14. The behavior determination system as claimed in claim 8, wherein
the analyzing the measurement includes
calculating, as the time parameter, a stance time in which the user stands on each foot, for each foot, and
calculating a two-leg support time in which the user stand on both feet, based on the stance time calculated for each foot, and
the detecting the peak point includes determining the user's behavior as a running behavior when the two-leg support time is less than the fourth predetermined value.

15. A behavior determination method to be implemented in an information processing device, comprising:
acquiring, by the information processing device, measurement data indicating a pressure or a force measured by one or a plurality of sensors provided on a sole surface of a user's foot;
analyzing, by the information processing device, the measurement data, to identify one load phase in which the user makes one step, and calculating a plantar pressure parameter and a time parameter for every one load phase; and
detecting, by the information processing device, a peak point where a maximum local maximum is obtained for every predetermined time, based on the plantar pressure parameter and the time parameter, and determining a behavior of the user based on the peak point.

16. The behavior determination method as claimed in claim 15, wherein
the plurality of sensors include at least one sensor provided at a rear foot portion on the sole surface, and at least one sensor provided at a front foot portion on is installed at least one at the rear portion and the front portion on the sole surface, and
the detecting the peak point includes comparing the measurement data of all of the sensors, and determining the user's behavior as a walking behavior when a total peak difference, that is a difference between a peak value in a first phase of the one load phase and a peak value of a second phase of the one load phase, is less than a third predetermined value.

17. The behavior determination method as claimed in claim 15, wherein
the plurality of sensors include at least one sensor provided at each of a front foot portion, a mid foot portion, and a rear foot portion on the sole surface, and
the detecting the peak point includes determining, based on the measurement data of each of the at least one sensor, the user's behavior as a stair climbing down behavior when a peak difference, that is a difference between a peak value in a first phase of the one load phase and a peak value of a second phase of the one load phase, is less than a first predetermined value, and the pressure or the force is concentrated at the front foot portion or the mid foot portion.

18. The behavior determination method as claimed in claim 15, wherein
the plurality of sensors are provided on the sole surface, and
the detecting the peak point includes comparing the measurement data of all of the sensors, and determining the user's behavior as a stair climbing up behavior when a total peak difference between a peak value in a first phase of the one load phase and a peak value in a second phase of the one load phase is greater than or equal to a second predetermined value, and the peak value in the second phase of the one load phase is larger than the peak value of the first phase of the one load phase.

19. A non-transitory computer-readable storage medium having stored therein a program which, when executed by a computer, causes the computer to perform a process comprising:
acquiring measurement data indicating a pressure or a force measured by one or a plurality of sensors provided on a sole surface of a user's foot;
analyzing the measurement data, to identify one load phase in which the user makes one step, and calculating a plantar pressure parameter and a time parameter for every one load phase; and
detecting a peak point where a maximum local maximum is obtained for every predetermined time, based on the plantar pressure parameter and the time parameter, and determining a behavior of the user based on the peak point.

20. The non-transitory compute-readable storage medium as claimed in claim 19, wherein
the plurality of sensors include at least one sensor provided at a rear foot portion on the sole surface, and at least one sensor provided at a front foot portion on is installed at least one at the rear portion and the front portion on the sole surface, and
the detecting the peak point includes comparing the measurement data of all of the sensors, and determining the user's behavior as a walking behavior when a total peak difference, that is a difference between a peak value in a first phase of the one load phase and a peak value of a second phase of the one load phase, is less than a third predetermined value.
